(19) United States
(12) Patent Application Publication
(10) Pub. No.: US 2021/0077492 A1
(43) Pub. Date: Mar. 18, 2021

(54) METHODS AND COMPOSITIONS OF TREATING PANCREATIC CANCER

(71) Applicant: The Trustees of Indiana University, Indianapolis, IN (US)

(72) Inventor: Francis Obunyakha Enane, Indianapolis, IN (US)

(21) Appl. No.: 17/023,820

(22) Filed: Sep. 17, 2020

Related U.S. Application Data
(60) Provisional application No. 62/901,298, filed on Sep. 17, 2019.

(51) Int. Cl. A61K 31/513; A61K 31/53; A61K 31/506; A61K 31/202; A61P 35/00

(52) U.S. Cl. CPC A61K 31/513; A61K 31/53; A61P 35/00; A61K 31/202; A61K 31/506

(57) ABSTRACT

A method of treating pancreatic cancer in a patient in need thereof according to an embodiment includes administering to the patient an effective amount of a pharmaceutical composition comprising NSC232003 (DT1), NSC127716 (DAC), and one or more pharmaceutically acceptable carriers, excipients, or diluents.

### CROSS REFERENCE TO RELATED APPLICATIONS

This application claims priority to U.S. Provisional Patent Application Ser. No. 62/901,298 filed on Sep. 17, 2019, which is incorporated by reference herein in its entirety.

### STATEMENT ON FEDERALLY SPONSORED RESEARCH OR DEVELOPMENT

This invention was made with government support under TR001108 awarded by the National Institutes of Health (NIH). The government has certain rights in the invention.

### BACKGROUND

Pancreatic cancer is a lethal human malignancy that is growing in global incidence and mortality. The majority of pancreatic cancer patients die within 6-12 months of diagnosis, and pancreatic cancer is resistant to current FDA approved therapies. Pancreatic ductal adenocarcinoma (PDAC) is metastatic and the most lethal form of pancreatic cancer, accounting for about 90% of the diagnoses. PDAC typically progresses even with treatment by multiple drugs (median survival 8.5 months), and the median progression free survival for progressive disease is 1.6-2.9 months. In the U.S, PDAC has a 5-year survival rate of about 8%, and PDAC is projected to be the second leading cause of cancer related mortality by 2020-2030.

Current therapies are not effective. One reason may be that molecular mechanisms of PDAC result in a high rate (about 80%) of genetic mutations (e.g., TP53 and p161CDKN2A) that confer resistance to current FDA approved treatments, including, for example, gemcitabine monotherapy or in combination with folfirinox (5-FU), irinotecan, oxaliplatin, or cisplatin, which may attempt to kill PDAC cells by inducing apoptosis. These molecular aspects of PDAC may alter apoptosis pathways that selectively remove the signaling pathways by which the master protein p-53 controls apoptosis, which may clash with the intent of most cancer treatments to activate apoptosis and induce cytotoxicity. The result may be that apoptosis of tumor cells does not occur. Another reason that current therapies are not effective is that treatments with non-p-53 targets (e.g., tyrosine kinases, metabolic enzymes, or radiation induced DNA-damage) may also work through apoptosis and, thus, contribute to refractoriness. Meanwhile, normal stem cells with intact apoptosis-pathways may be destroyed, causing significant toxicities to diminish quality of remaining life.

Cell differentiation is a dominant and evolutionarily-conserved method to physiologically regulate normal cell growth and division, enhance cell maturation, and form non-proliferative and homeostasis cell types. Therefore, loss of differentiation is essential to cancer cell proliferation and survival. PDAC cells are poorly differentiated and the underlying processes of how differentiation is lost is mostly unknown. The growth of PDAC cells depends on KRAS mutations that stabilize the Myc oncogenes resulting in unregulated replication.

A non-cytotoxic therapy that physiologically regulates cell proliferation independent of apoptosis, such as cell differentiation therapy, and that targets unregulated differentiation but spares the normal cell self-replication required for homeostasis, by activation of pancreas specific tumor suppressor genes (NKX6-1, HNF6, AMY2A, AMY2B) is needed.

### SUMMARY

According to an embodiment, a method of treating pancreatic cancer in a patient in need thereof includes administering to the patient an effective amount of a pharmaceutical composition comprising NSC232003 (DT1), NSC127716 (DAC), and one or more pharmaceutically acceptable carriers, excipients, or diluents.

In some embodiments, the method further includes administering NSC112907 (THU) to the patient.

In some embodiments, the route of administration is parenteral.

In some embodiments, the patient is a human.

In some embodiments, the method further includes administering to the patient at least one additional active ingredient.

In some embodiments, the amount of DT1 administered to the patient is from about 0.1 to about 10 mg of DT1 per kg of patient body weight.

In some embodiments, the amount of DAC administered to the patient is from about 0.1 to about 1 mg of DAC per kg of patient body weight.

In some embodiments, the amount of THU administered to the patient is from about 10 to about 40 mg of THU per kg of patient body weight.

In some embodiments, the patient is administered from about 0.1 to about 10 mg of DT1 per kg of patient body weight and from about 0.1 to about 1 mg of DAC per kg of patient body weight.

In some embodiments, the patient is administered from about 0.1 to about 10 mg of DT1 per kg of patient body weight, from about 0.1 to about 1 mg of DAC per kg of patient body weight, and from about 10 to about 40 mg of THU per kg of patient body weight.

In some embodiments, the pancreatic cancer is pancreatic ductal cell adenocarcinoma (PDAC).

According to another embodiment, a method of treating pancreatic cancer in a patient in need thereof includes administering to the patient an effective amount of an inhibitor of UHRF1 and an inhibitor of DNMT1.

In some embodiments, the method further includes administering an inhibitor of cytidine deaminase (CDA).

In some embodiments, the pancreatic cancer is pancreatic ductal cell adenocarcinoma (PDAC).

According to another embodiment, a method of treating pancreatic cancer in a patient in need thereof includes administering to the patient an effective amount of a pharmaceutical composition comprising NSC127716 (DAC), NSC122758 (ATRA), and one or more pharmaceutically acceptable carriers, excipients, or diluents.

In some embodiments, the route of administration is parenteral.

In some embodiments, the patient is a human.

In some embodiments, the amount of ATRA administered to the patient is from about 1 to about 100 mg of ATRA per kg of patient body weight.

In some embodiments, the amount of DAC administered to the patient is from about 0.1 to about 1 mg of DAC per kg of patient body weight.

In some embodiments, the pancreatic cancer is pancreatic ductal cell adenocarcinoma (PDAC).

This summary is not intended to identify key or essential features of the claimed subject matter, nor is it intended to be used as an aid in limiting the scope of the claimed subject matter. Further embodiments, forms, features, and aspects of the present application shall become apparent from the description and figures provided herewith.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The concepts described herein are illustrative by way of example and not by way of limitation in the accompanying figures. For simplicity and clarity of illustration, elements illustrated in the figures are not necessarily drawn to scale. Where considered appropriate, reference labels have been repeated among the figures to indicate corresponding or analogous elements.

FIG. 1 is a diagram illustrating a model hypothesis according to an embodiment;

FIG. 2 is a diagram illustrating oncogenic cooperation of DNA methylation-corepressors according to an embodiment;

FIG. 3 is a graph illustrating UHRF1 expression in PDAC according to an embodiment;

FIG. 4 is a graph illustrating DNMT1 expression in PDAC according to an embodiment;

FIG. 5 is a graph illustrating expression of transcriptional corepressor of the retinoic acid receptor pathway TBL1XR1 in PDAC according to an embodiment;

FIG. 6 is a graph illustrating UHRF1 expression levels in KRAS and TP53 mutant PDAC patients according to an embodiment;

FIG. 7 is a graph illustrating expression of pancreas differentiation and homeostasis genes in PDAC with high versus low UHRF1 levels according to an embodiment;

FIG. 8 is a graph illustrating pancreas differentiation and homeostasis genes in PDAC with high versus low DNMT1 levels according to an embodiment;

FIG. 9 is a graph illustrating pancreas differentiation and homeostasis genes in PDAC with high versus low DNMT1 levels according to an embodiment;

FIG. 10 is a graph of methylation levels of pancreas differentiation and homeostasis genes according to an embodiment;

FIG. 11 is a microarray data illustration showing expression of pancreas differentiation and homeostasis genes in matched PDAC versus non-malignant pancreas according to an embodiment;

FIG. 12 is graphs illustrating examples of pancreas specific markers in non-malignant versus PDAC samples according to an embodiment;

FIG. 13 is graphs illustrating inactivation of UHRF1 and DNMT1 by short-hairpin RNA (shRNA) according to an embodiment;

FIG. 14 is an illustration showing UHRF1 and DNMT1 shRNA knockdown according to an embodiment;

FIG. 15 is a graph illustrating inactivation of UHRF1 and DNMT1 decreasing pancreatic cancer proliferation according to an embodiment;

FIG. 16 is a graph illustrating that inactivation of UHRF1 and DNMT1 decreases pancreatic cancer proliferation according to an embodiment;

FIG. 17 is a graph illustrating that inactivation of UHRF1 decreases global DNA methylation in pancreatic cancer cells according to an embodiment;

FIG. 18 is an illustration of a small molecule compound NSC232003 (DT1) targeting SRA domain of UHRF1 according to an embodiment;

FIG. 19 is an illustration of six novel derivatives of NSC232003 (DT1) targeting SRA domain of UHRF1 with stronger potency designed using structure activity relationship (SAR) according to an embodiment;

FIG. 20 is an illustration of a small molecule compound decitabine (DAC) targeting DNMT1 according to an embodiment;

FIG. 21 is an illustration of a small molecule compound tetrahydrouridine (THU) targeting CDA according to an embodiment;

FIG. 22 is an illustration of a small molecule ATRA targeting retinoic acid receptor pathway according to an embodiment;

FIG. 23 is graphs illustrating that DT1 treatment activates pancreas specific tumor suppressor and differentiation factors according to an embodiment;

FIG. 24 is giemsa stains illustrating that combination therapy of DT1 and DAC induces differentiation based morphological changes according to an embodiment;

FIG. 25 is giesma stains illustrating that combination therapy of DAC and ATRA induces differentiation based morphological changes according to an embodiment;

FIG. 26 is a graph illustrating that targeting UHRF1 by DT1 decreases global DNA methylation in pancreatic cancer cells according to an embodiment;

FIG. 27 is a graph illustrating that synergistic DT-DAC combination therapy decreases PDAC growth according to an embodiment;

FIG. 28 is a graph illustrating that synergistic DT-DAC combination therapy decreases PDAC growth according to an embodiment;

FIG. 29 is a graph illustrating that synergistic DT-DAC combination therapy decreases PDAC growth according to an embodiment;

FIG. 30 is a heat map illustrating PDAC orthotopic Xenografts for therapeutic validation according to an embodiment;

FIG. 31 is a graph illustrating improved efficacy of Decitabine upon THU treatment in the orthotopic PDAC xenograft model using AsPc-1 cells according to an embodiment;

FIG. 32 is graphs illustrating differentiation induction and suppression of pancreatic cancer cells treated with DAC+ATRA according to an embodiment;

FIG. 33 is an illustration of a biotin-DT1 conjugate according to an embodiment;

FIG. 34 is an illustration of DT1 immobilized to agarose beads according to an embodiment;

FIG. 35 is an illustration showing an analysis of hematoxylin and eosin-stained KPC mixed-gender mouse models (Jackson Laboratory) compared to wild-type (WT) according to an embodiment;

FIG. 36 is a chart illustrating gene expression RNA-seq levels of various pancreas differentiation markers compared to UHRF1 and DNMT1 in the KPC mouse model vs. age matched WT according to an embodiment; and

FIG. 37 is an illustration of UHRF1 and DNMT1 protein in the aggressive KPC model (KRAS and TP53) vs. less aggressive KC model (KRAS only) according to an embodiment.

### DETAILED DESCRIPTION

Although the concepts of the present disclosure are susceptible to various modifications and alternative forms, specific embodiments have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. It should be further appreciated that although reference to a “preferred” component or feature may indicate the desirability of a particular component or feature with respect to an embodiment, the disclosure is not so limiting with respect to other embodiments, which may omit such a component or feature. Further, when a particular feature, structure, or characteristic is described in connection with an embodiment, it is submitted that it is within the knowledge of one skilled in the art to implement such feature, structure, or characteristic in connection with other embodiments whether or not explicitly described. Additionally, it should be appreciated that items included in a list in the form of “at least one of A, B, and C” can mean (A); (B); (C); (A and B); (B and C); (A and C); or (A, B, and C). Similarly, items listed in the form of “at least one of A, B, or C” can mean (A); (B); (C); (A and B); (B and C); (A and C); or (A, B, and C). Further, with respect to the claims, the use of words and phrases such as “a,” “an,” “at least one,” and/or “at least one portion” should not be interpreted so as to be limiting to only one such element unless specifically stated to the contrary, and the use of phrases such as “at least a portion” and/or “a portion” should be interpreted as encompassing both embodiments including only a portion of such element and embodiments including the entirety of such element unless specifically stated to the contrary.

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures unless indicated to the contrary. Additionally, the inclusion of a structural or method feature in a particular figure is not meant to imply that such feature is required in all embodiments and, in some embodiments, may not be included or may be combined with other features.

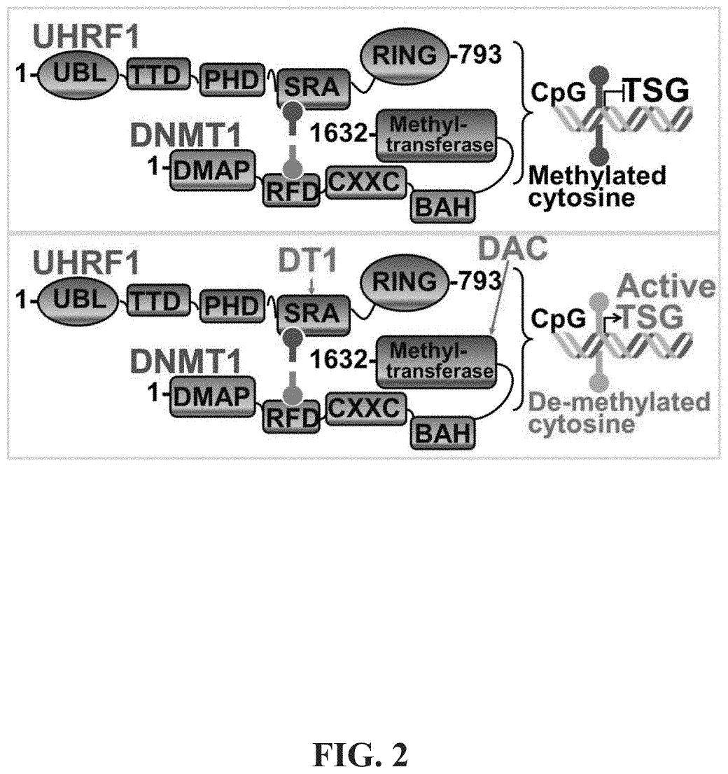

The ability of corepressor enzymes involved in gene repression to provide PDAC therapy is disclosed. Two such key reprogramming enzymes, Ubiquitin as with PHD and Ring Finger Domains 1 (UHRF1) and DNA Methyl-transferase-1 (DNMT1), which are shown in FIG. 2, are critical in epigenetically repressing differentiation genes thus altering differentiation-based cell cycle exit in PDAC.

UHRF1 is a transcriptional repressor that cooperates with DNA methyl transferase 1 (DNMT1) to modify DNA methylation. DNA methylation epigenetically suppresses multiple tumor suppressor genes (TSG). The therapeutic potential of targeting UHRF1 and DNMT1 epigenetic activities is not clearly delineated.

PDAC cases with mutations in KRAS and TP53 genes express high levels of UHRF1; upregulation of UHRF1 predicted poor patient survival (p<0.0009, n=182). Oncogenic cooperation between UHRF1 and DNMT1 suppresses multiple tumor suppressor genes (TSG). Such combinatorial, non-cytotoxic, epigenetic therapy targets both UHRF1 and DNMT1 in PDAC.

The present disclosure provides a method of treating pancreatic cancer in a patient in need thereof. In some embodiments, the method comprises administering to the patient an effective amount of a pharmaceutical composition comprising NSC232003 (DT1), NSC127716 (DAC), and one or more pharmaceutically acceptable carriers, excipients, or diluents. In some embodiments, the method is an epigenetic therapy used to activate tumor suppressor genes. In some embodiments, the method results in minimal toxicity to non-PDAC cells. In some embodiments, the method is apoptosis independent.

The present disclosure also provides a method of treating pancreatic cancer in a patient in need thereof. In some embodiments, the method comprises administering to the patient an effective amount of a pharmaceutical composition comprising NSC127716 (DAC), NSC122758 (ATRA), and one or more pharmaceutically acceptable carriers, excipients, or diluents. In some embodiments, the cancer is pancreatic ductal cell adenocarcinoma (PDAC).

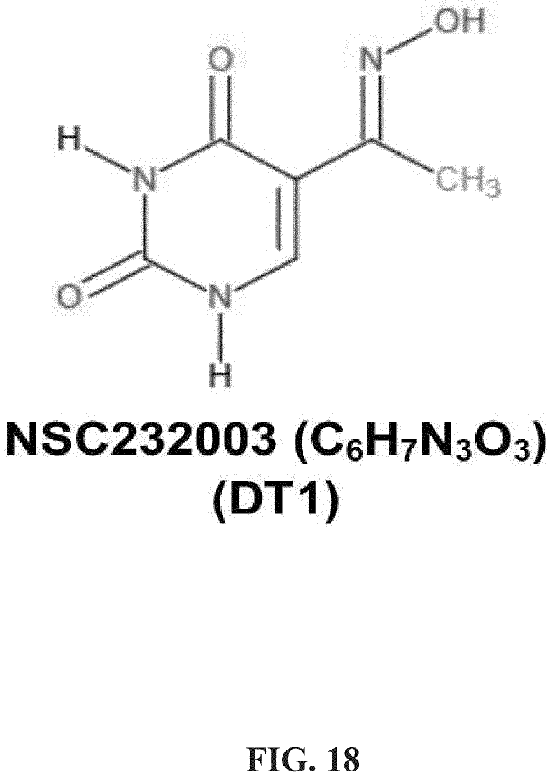

NSC232003 is also referred to herein as DT1 (DT Therapeutics, LLC). The molecular formula of DT1 is C₆H₇N₃O₃. DT1 has CAS No. 1905453-18-0. The chemical structure of DT1 is illustrated in FIG. 18. DT1 (DT Therapeutics, LLC) is a uracil derivative. DT1 modulates and binds to the SET and RING (SRA) domain of the enzyme UHRF1, and DT1 may decrease global DNA methylation.

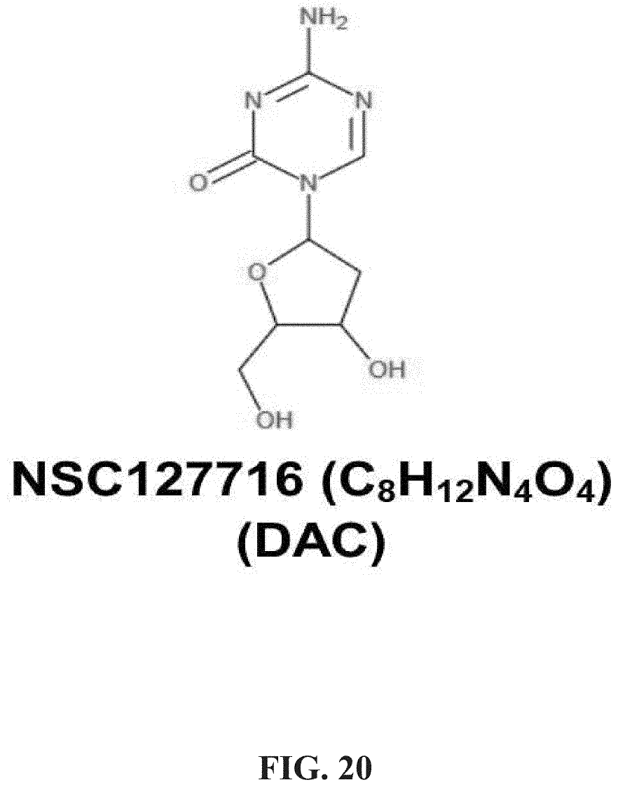

NSC127716 is also referred to herein as decitabine or DAC. The molecular formula of DAC is C₈H₁₂N₄O₄. DAC has CAS No. 2353-33-5. The chemical structure of DAC is illustrated in FIG. 20. Decitabine (DAC, DT Therapeutics, LLC) is a cytidine derivative that degrades DNMT1.

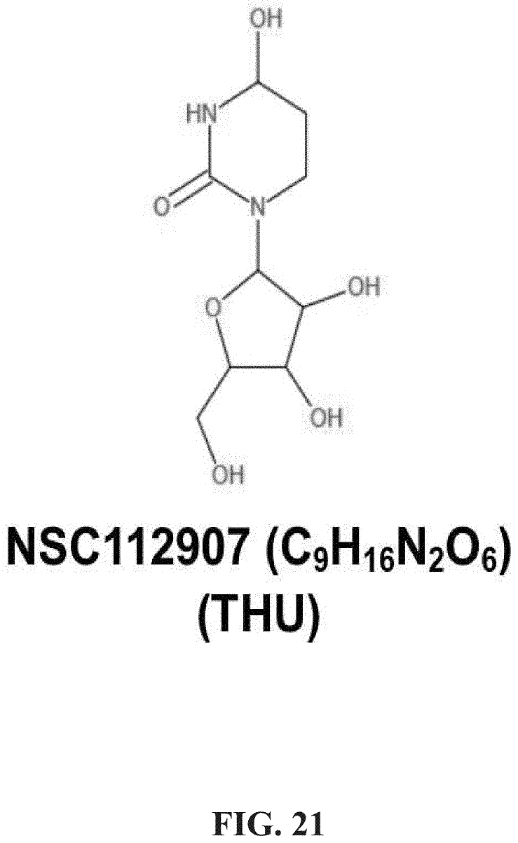

In some embodiment, the methods of the present disclosure further comprise administering NSC112907 (THU) to the patient in need thereof. NSC112907 is also referred to herein as tetrahydrouridine or THU. The molecular formula of THU is C₉H₁₆N₂O₆. THU has CAS No. 18771-50-1. The chemical structure of THU is illustrated in FIG. 21. THU inhibits cytidine deaminase (CDA).

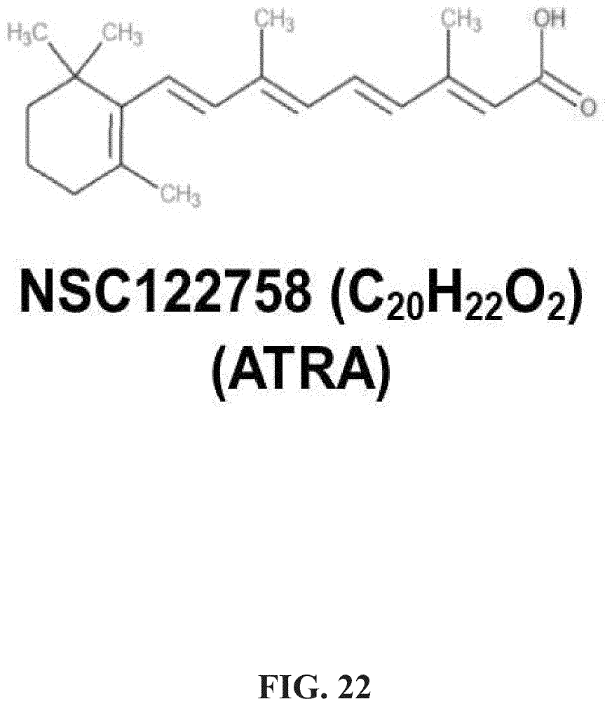

NSC122758 is also referred to herein as all-trans-retinoic-acid or ATRA. The molecular formula of ATRA is C₂H₂₂O₂. The chemical structure of ATRA is illustrated in FIG. 22. ATRA induces differentiation by disrupting RAR-CoR such as TBL1XR1.

DT1, DAC, THU, and/or ATRA are also referred to herein as a compound of the present disclosure. DT1, DAC, THU, and/or ATRA are described with reference to the specific compounds illustrated herein. In addition, DT1, DAC, THU, and/or ATRA may exist in any number of different forms or derivatives, all within the scope of the present disclosure. Alternative forms or derivatives, include, for example, pharmaceutically acceptable salts, prodrugs and active metabolites, tautomers, and solid forms, including without limitation different crystal forms, polymorphic or amorphous solids, including hydrates and solvates thereof, and other forms.

Unless specified to the contrary, specification of DT1, DAC, THU, and/or ATRA herein includes pharmaceutically acceptable salts of such compounds. Thus, DT1, DAC, THU, and/or ATRA can be in the form of pharmaceutically acceptable salts or can be formulated as pharmaceutically acceptable salts. Contemplated pharmaceutically acceptable salt forms of the present disclosure include, without limitation, mono, bis, tris, tetrakis, and so on. Pharmaceutically acceptable salts of the present disclosure are non-toxic in the amounts and concentrations at which such pharmaceutically acceptable salts are administered. The preparation of such pharmaceutically acceptable salts of the present disclosure can facilitate the pharmacological use by altering the physical characteristics of a compound of the present disclosure without preventing it from exerting its physiological effect.

As used herein, the term “pharmaceutically acceptable,” with respect to salts and composition components such as carriers, excipients, and diluents, refers to those salts and components which are not deleterious to a patient and which are compatible with other ingredients, active ingredients, salts, or components. Pharmaceutically acceptable includes “veterinarily acceptable,” and thus includes both human and non-human mammal applications independently.

As used herein, the term “pharmaceutically acceptable salt” refers to salts commonly used to form alkali metal salts and to form additional salts of free acids or free bases. Such salts include, for example, the physiologically acceptable salts listed in Handbook of Pharmaceutical Salts: Properties, Selection and Use, P. H. Stahl and C. G. Wermuth (Eds.), Wiley-VCH, New York, 2002. Salt formation can occur at one or more positions having labile protons. The pharmaceutically acceptable salts of a compound of the present disclosure include both acid addition salts and base addition salts.

As used herein, the term “pharmaceutical composition” refers to a pharmaceutical preparation that contains a compound of the present disclosure, or a pharmaceutically acceptable salt thereof, and is suitable for administration to a patient for therapeutic purposes. As used herein, the term “patient” refers to a living organism that is treated with a compound of the present disclosure, including without limitation any mammal such as, for example, humans, other primates (e.g., monkeys, chimpanzees, etc.), companion animals (e.g., dogs, cats, horses, etc.), farm animals (e.g., goats, sheep, pigs, cattle, etc.), laboratory animals (e.g., mice, rats, etc.), and wild and zoo animals (e.g., wolves, bears, deer, etc.).

In some embodiments, the pharmaceutical composition may include at least one pharmaceutically acceptable component to provide an improved formulation of a compound of the present disclosure, including without limitation one or more pharmaceutically acceptable carriers, excipients or diluents. The carrier, excipient or diluent may take a wide variety of forms depending on the form of preparation desired for administration.

As used herein, the term “carrier” includes without limitation calcium carbonate, calcium phosphate, various sugars, such as lactose, glucose, or sucrose, types of starch, cellulose derivatives, gelatin, lipids, liposomes, nanoparticles, physiologically acceptable liquids as solvents or for suspensions, including, for example, sterile solutions of water for injection (WFI), saline solution, dextrose solution, Hank's solution, Ringer's solution, vegetable oils, mineral oils, animal oils, polyethylene glycols, liquid paraffin, and the like.

As used herein, the term “excipient” generally includes without limitation fillers, binders, disintegrants, glidants, lubricants, complexing agents, solubilizers, stabilizer, preservatives, and surfactants, which may be chosen to facilitate administration of the compound by a particular route. Suitable excipients may also include, for example, colloidal silicon dioxide, silica gel, talc, magnesium silicate, calcium silicate, sodium aluminosilicate, magnesium trisilicate, powdered cellulose, macrocrystalline cellulose, carboxymethyl cellulose, crosslinked sodium carboxymethylcellulose, sodium benzoate, calcium carbonate, magnesium carbonate, stearic acid, aluminum stearate, calcium stearate, magnesium stearate, zinc stearate, sodium stearyl fumarate, syloid, stearowet C, magnesium oxide, starch, sodium starch glycolate, glyceryl monostearate, glyceryl dibehenate, glyceryl palmitostearate, hydrogenated vegetable oil, hydrogenated cotton seed oil, castor seed oil, mineral oil, polyethylene glycol (e.g., PEG 4000-8000), polyoxyethylene glycol, poloxamers, povidone, crospovidone, croscarmellose sodium, alginic acid, casein, methacrylic acid divinylbenzene copolymer, sodium docusate, cyclodextrins (e.g., 2-hydroxypropyl-delta-cyclodextrin), polysorbates (e.g., polysorbate 80), cetrimide, TPGS (d-alpha-tocopheryl polyethylene glycol 1000 succinate), magnesium lauryl sulfate, sodium lauryl sulfate, polyethylene glycol ethers, di-fatty acid ester of polyethylene glycols, or a polyoxyalkylene sorbitan fatty acid ester (e.g., polyoxyethylene sorbitan ester Tween®), polyoxyethylene sorbitan fatty acid esters, sorbitan fatty acid ester, e.g., a sorbitan fatty acid ester from a fatty acid such as oleic, stearic or palmitic acid, mannitol, xylitol, sorbitol, maltose, lactose, lactose monohydrate or lactose spray dried, sucrose, fructose, calcium phosphate, dibasic calcium phosphate, tribasic calcium phosphate, calcium sulfate, dextrates, dextran, dextrin, dextrose, cellulose acetate, maltodextrin, simethicone, polydextrosem, chitosan, gelatin, HPMC (hydroxypropyl methyl celluloses), HPC (hydroxypropyl cellulose), hydroxyethyl cellulose, etc.

It should be appreciated that any diluent known in the art may be utilized in accordance with the present disclosure. In some embodiments of the present disclosure, the diluent is water soluble. In some embodiments of the present disclosure, the diluent is water insoluble. As used herein, the term “diluent” includes without limitation water, saline, phosphate buffered saline (PBS), dextrose, glycerol, ethanol, buffered sodium or ammonium acetate solution, or the like, and combinations thereof.

In some embodiments, the pharmaceutical compositions of the present disclosure include at least one additional active ingredient. As used herein, the term “active ingredient” refers to a therapeutically active compound, as well as any prodrugs thereof and pharmaceutically acceptable salts, hydrates, and solvates of the compound and the prodrugs. Additional active ingredients may be combined with a compound of the present disclosure and may be either administered separately or in the same pharmaceutical composition. It should be appreciated that the amount of additional active ingredients to be given may be determined by one skilled in the art based upon therapy with a compound of the present disclosure.

In some embodiments, the pharmaceutical composition is a human pharmaceutical composition. As used herein, the term “human pharmaceutical composition” refers to a pharmaceutical composition intended for administration to a human.

The pharmaceutical compositions of the present disclosure are suitable for administration to a patient by any suitable means, including without limitation those means used to administer conventional antimicrobials. It should be appreciated that the pharmaceutical compositions of the present disclosure may be administered using any applicable route that would be considered by one of ordinary skill, including without limitation oral, parenteral, intravenous (“IV”) injection or infusion, intravesical, subcutaneous (“SC”), intramuscular (“IM”), intraperitoneal, intradermal, intraocular, inhalation (and intrapulmonary), intranasal, transdermal, epicutaneously, subdermal, topical, mucosal, nasal, ophthalmic, impression into skin, intravaginal, intrauterine, intracervical, and rectal. Such dosage forms should allow a compound of the present disclosure to reach target cells. Other factors are well known in the art and include considerations such as toxicity and dosage forms that retard a compound or composition from exerting its effects. Techniques and formulations generally may be found in Remington: The Science and Practice of Pharmacy, 21st edition, Lippincott, Williams and Wilkins, Philadelphia, Pa., 2005.

As used herein, the terms “treating,” “treatment,” “therapy,” and like terms refer to administration of a compound or pharmaceutical composition of the present disclosure in an amount effective to prevent, alleviate or ameliorate one or more symptoms of a disease or condition (i.e., indication) and/or to prolong the survival of the patient being treated. In some embodiments, “treating,” “treatment,” “therapy,” and like terms also include without limitation reducing or eliminating pancreatic cancer (e.g., PDAC) in a patient.

In carrying out the methods of the present disclosure, an effective amount of a compound of the present disclosure is administered to a patient in need thereof. As used herein, the term “effective amount,” in the context of administration, refers to the amount of a compound or pharmaceutical composition of the present disclosure that when administered to a patient is sufficient to prevent, alleviate or ameliorate one or more symptoms of a disease or condition (i.e., indication) and/or to prolong the survival of the patient being treated. Such an amount should result in no or few adverse events in the treated patient. Similarly, such an amount should result in no or few toxic effects in the treated patient. It should be appreciated that the amount of a compound or pharmaceutical composition of the present disclosure will vary depending upon a number of factors, including without limitation the activity of a compound of the present disclosure (in vitro, e.g. a compound of the present disclosure vs. target, or in vivo activity in animal efficacy models), pharmacokinetic results in animal models (e.g., biological half-life or bioavailability), the type of patient being treated, the patient's age, size, weight, and general physical condition, the disorder associated with the patient, and the dosing regimen being employed in the treatment.

In some embodiments of the present disclosure, an effective amount of a compound of the present disclosure to be delivered to a patient in need thereof can be quantified by determining micrograms of a compound of the present disclosure per kilogram of patient body weight. In some embodiments, the amount of a compound of the present disclosure administered to a patient is from about 0.1 to about 1000 milligram (mg) of a compound of the present disclosure per kilogram (kg) of patient body weight. In some embodiments, the amount of a compound of the present disclosure administered to a patient is from about 0.1 to about 500 mg of a compound of the present disclosure per kg of patient body weight. In some embodiments, the amount of a compound of the present disclosure administered to a patient is from about 0.1 to about 300 mg of a compound of the present disclosure per kg of patient body weight. In some embodiments, the amount of a compound of the present disclosure administered to a patient is from about 0.1 to about 200 mg of a compound of the present disclosure per kg of patient body weight. In some embodiments, the amount of a compound of the present disclosure administered to a patient is from about 0.1 to about 100 mg of a compound of the present disclosure per kg of patient body weight. In some embodiments, the amount of a compound of the present disclosure administered to a patient is from about 0.1 to about 50 mg of a compound of the present disclosure per kg of patient body weight. In some embodiments, the amount of a compound of the present disclosure administered to a patient is from about 0.1 to about 40 mg of a compound of the present disclosure per kg of patient body weight. In some embodiments, the amount of a compound of the present disclosure administered to a patient is from about 0.1 to about 30 mg of a compound of the present disclosure per kg of patient body weight. In some embodiments, the amount of a compound of the present disclosure administered to a patient is from about 0.1 to about 20 mg of a compound of the present disclosure per kg of patient body weight. In some embodiments, the amount of a compound of the present disclosure administered to a patient is from about 0.1 to about 10 mg of a compound of the present disclosure per kg of patient body weight. In some embodiments, the amount of a compound of the present disclosure administered to a patient is from about 0.1 to about 5 mg of a compound of the present disclosure per kg of patient body weight. In some embodiments, the amount of a compound of the present disclosure administered to a patient is from about 0.1 to about 4 mg of a compound of the present disclosure per kg of patient body weight. In some embodiments, the amount of a compound of the present disclosure administered to a patient is from about 0.1 to about 3 mg of a compound of the present disclosure per kg of patient body weight. In some embodiments, the amount of a compound of the present disclosure administered to a patient is from about 0.1 to about 2 mg of a compound of the present disclosure per kg of patient body weight. In some embodiments, the amount of a compound of the present disclosure administered to a patient is from about 0.1 to about 1 mg of a compound of the present disclosure per kg of patient body weight. As those of ordinary skill in the art understand multiple doses may be used.

In some embodiments, the amount of DT1 administered to a patient is from about 0.1 to about 50 milligram (mg) of DT1 per kilogram (kg) of patient body weight. In some embodiments, the amount of DT1 administered to a patient is from about 0.1 to about 40 milligram (mg) of DT1 per kilogram (kg) of patient body weight. In some embodiments, the amount of DT1 administered to a patient is from about 0.1 to about 30 milligram (mg) of DT1 per kilogram (kg) of patient body weight. In some embodiments, the amount of DT1 administered to a patient is from about 0.1 to about 20 milligram (mg) of DT1 per kilogram (kg) of patient body weight. In some embodiments, the amount of DT1 administered to a patient is from about 0.1 to about 10 milligram (mg) of DT1 per kilogram (kg) of patient body weight. In some embodiments, the amount of DT1 administered to a patient is from about 0.1 to about 5 milligram (mg) of DT1 per kilogram (kg) of patient body weight. In some embodiments, the amount of DT1 administered to a patient is from about 5 to about 30 milligram (mg) of DT1 per kilogram (kg) of patient body weight. In some embodiments, the amount of DT1 administered to a patient is from about 0.1 to about 1 milligram (mg) of DT1 per kilogram (kg) of patient body weight. In some embodiments, the amount of DT1 administered to a patient is from about 0.1 to about 0.2 milligram (mg) of DT1 per kilogram (kg) of patient body weight.

In some embodiments, the amount of DAC administered to a patient is from about 0.1 to about 20 milligram (mg) of DAC per kilogram (kg) of patient body weight. In some embodiments, the amount of DAC administered to a patient is from about 0.1 to about 15 milligram (mg) of DAC per kilogram (kg) of patient body weight. In some embodiments, the amount of DAC administered to a patient is from about 0.1 to about 10 milligram (mg) of DAC per kilogram (kg) of patient body weight. In some embodiments, the amount of DAC administered to a patient is from about 0.1 to about 5 milligram (mg) of DAC per kilogram (kg) of patient body weight. In some embodiments, the amount of DAC administered to a patient is from about 0.1 to about 4 milligram (mg) of DAC per kilogram (kg) of patient body weight. In some embodiments, the amount of DAC administered to a patient is from about 0.1 to about 3 milligram (mg) of DAC per kilogram (kg) of patient body weight. In some embodiments, the amount of DAC administered to a patient is from about 0.1 to about 2 milligram (mg) of DAC per kilogram (kg) of patient body weight. In some embodiments, the amount of DAC administered to a patient is from about 0.1 to about 1 milligram (mg) of DAC per kilogram (kg) of patient body weight. In some embodiments, the amount of DAC administered to a patient is from about 0.1 to about 0.9 milligram (mg) of DAC per kilogram (kg) of patient body weight. In some embodiments, the amount of DAC administered to a patient is from about 0.1 to about 0.8 milligram (mg) of DAC per kilogram (kg) of patient body weight. In some embodiments, the amount of DAC administered to a patient is from about 0.1 to about 0.7 milligram (mg) of DAC per kilogram (kg) of patient body weight. In some embodiments, the amount of DAC administered to a patient is from about 0.1 to about 0.6 milligram (mg) of DAC per kilogram (kg) of patient body weight. In some embodiments, the amount of DAC administered to a patient is from about 0.1 to about 0.5 milligram (mg) of DAC per kilogram (kg) of patient body weight. In some embodiments, the amount of DAC administered to a patient is from about 0.1 to about 0.4 milligram (mg) of DAC per kilogram (kg) of patient body weight. In some embodiments, the amount of DAC administered to a patient is from about 0.1 to about 0.3 milligram (mg) of DAC per kilogram (kg) of patient body weight. In some embodiments, the amount of DAC administered to a patient is from about 0.1 to about 0.2 milligram (mg) of DAC per kilogram (kg) of patient body weight.

In some embodiments, the amount of THU administered to a patient is from about 0.1 to about 100 milligram (mg) of THU per kilogram (kg) of patient body weight. In some embodiments, the amount of THU administered to a patient is from about 0.1 to about 50 milligram (mg) of THU per kilogram (kg) of patient body weight. In some embodiments, the amount of THU administered to a patient is from about 0.1 to about 40 milligram (mg) of THU per kilogram (kg) of patient body weight. In some embodiments, the amount of THU administered to a patient is from about 0.1 to about 30 milligram (mg) of THU per kilogram (kg) of patient body weight. In some embodiments, the amount of THU administered to a patient is from about 0.1 to about 20 milligram (mg) of THU per kilogram (kg) of patient body weight. In some embodiments, the amount of THU administered to a patient is from about 0.1 to about 10 milligram (mg) of THU per kilogram (kg) of patient body weight. In some embodiments, the amount of THU administered to a patient is from about 10 to about 50 milligram (mg) of THU per kilogram (kg) of patient body weight. In some embodiments, the amount of THU administered to a patient is from about 10 to about 40 milligram (mg) of THU per kilogram (kg) of patient body weight. In some embodiments, the amount of THU administered to a patient is from about 10 to about 30 milligram (mg) of THU per kilogram (kg) of patient body weight. In some embodiments, the amount of THU administered to a patient is from about 10 to about 20 milligram (mg) of THU per kilogram (kg) of patient body weight. In some embodiments, the amount of THU administered to a patient is from about 20 to about 40 milligram (mg) of THU per kilogram (kg) of patient body weight. In some embodiments, the amount of THU administered to a patient is from about 5 to about 15 milligram (mg) of THU per kilogram (kg) of patient body weight. In some embodiments, the amount of THU administered to a patient is from about 5 to about 20 milligram (mg) of THU per kilogram (kg) of patient body weight.

In some embodiments, the amount of ATRA administered to a patient is from about 0.1 to about 1000 milligram (mg) of ATRA per kilogram (kg) of patient body weight. In some embodiments, the amount of ATRA administered to a patient is from about 0.1 to about 500 mg of ATRA per kg of patient body weight. In some embodiments, the amount of ATRA administered to a patient is from about 0.1 to about 300 mg of ATRA per kg of patient body weight. In some embodiments, the amount of ATRA administered to a patient is from about 0.1 to about 200 mg of ATRA per kg of patient body weight. In some embodiments, the amount of ATRA administered to a patient is from about 0.1 to about 100 mg of ATRA per kg of patient body weight. In some embodiments, the amount of ATRA administered to a patient is from about 0.1 to about 50 mg of ATRA per kg of patient body weight. In some embodiments, the amount of ATRA administered to a patient is from about 0.1 to about 40 mg of ATRA per kg of patient body weight. In some embodiments, the amount of ATRA administered to a patient is from about 0.1 to about 30 mg of ATRA per kg of patient body weight. In some embodiments, the amount of ATRA administered to a patient is from about 0.1 to about 20 mg of ATRA per kg of patient body weight. In some embodiments, the amount of ATRA administered to a patient is from about 0.1 to about 10 mg of ATRA per kg of patient body weight. In some embodiments, the amount of ATRA administered to a patient is from about 0.1 to about 5 mg of ATRA per kg of patient body weight. In some embodiments, the amount of ATRA administered to a patient is from about 0.1 to about 4 mg of ATRA per kg of patient body weight. In some embodiments, the amount of ATRA administered to a patient is from about 0.1 to about 3 mg of ATRA per kg of patient body weight. In some embodiments, the amount of ATRA administered to a patient is from about 0.1 to about 2 mg of ATRA per kg of patient body weight. In some embodiments, the amount of ATRA administered to a patient is from about 0.1 to about 1 mg of ATRA per kg of patient body weight.

In some embodiments of the present disclosure, a compound of the present disclosure is administered as a multiple dose regimen. As used herein, the term “multiple dose regimen” refers to a treatment time period of more than one day. In some embodiments of the present disclosure, the multiple dose regimen is a time period of up to about 2 days. In some embodiments of the present disclosure, the multiple dose regimen is a time period of up to about 3 days. In some embodiments of the present disclosure, the multiple dose regimen is a time period of up to about 4 days. In some embodiments of the present disclosure, the multiple dose regimen is a time period of up to about 5 days. In some embodiments of the present disclosure, the multiple dose regimen is a time period of up to about 6 days. In some embodiments of the present disclosure, the multiple dose regimen is a time period of up to about 7 days. In some embodiments of the present disclosure, the multiple dose regimen is a time period of up to about 14 days. In some embodiments of the present disclosure, the multiple dose regimen is a time period of up to about one month. In some embodiments of the present disclosure, the multiple dose regimen is a time period of up to about two months. In some embodiments of the present disclosure, the multiple dose regimen is a time period of up to about three months. In some embodiments of the present disclosure, the multiple dose regimen is a time period of up to about four months. In some embodiments of the present disclosure, the multiple dose regimen is a time period of up to about five months. In some embodiments of the present disclosure, the multiple dose regimen is a time period of up to about six months. Other time periods may be used herein.

The present disclosure also provides epigenetic therapy for pancreatic ductal cell adenocarcinoma (PDAC). The therapy comprises a composition that reactivates pancreas homeostasis and tumor suppressor genes without cytotoxicity to non-cancerous cells.

The present disclosure also provides the use of DT1 as a differentiation promoting agent therapy for PDAC.

The present disclosure also provides a method of treating cancer by administering at least one agent that regulates epigenetic modifying enzymes. In some embodiments, the agent is the enzyme UHRF1. In some embodiments, the agent mediates the actions of pancreas specific transcription factors but does not regulate the transcription factors themselves.

### EXAMPLES

Examples related to the present disclosure are described below. In some embodiments, alternative techniques can be used. The examples are intended to be illustrative and are not limiting or restrictive of the scope of the invention as set forth in the claims.

As shown in the below examples, treatment of patient derived pancreatic cancer cells (ASPC1) with DT1 decreased global DNA methylation. The combination of DT1 and DAC synergistically suppressed PDAC proliferation. Because DT1 and DAC are prone to metabolism by pyrimidine enzymes (e.g., cytidine deaminase (CDA)), the CDA inhibitor tetrahydrouridine (THU) did not inhibit PDAC proliferation alone. However, the combination of THU with DT1, DAC, or DT1 and DAC potently suppressed proliferation by a remarkable synergistic effect leading to a substantial reduction in the amount of DT1 and DAC needed to suppress PDAC growth. This low dose combination therapy minimized toxicity to allow a margin of safety needed to use non-apoptosis inducing therapy. Therefore, only a small amount of epigenetic drug(s) is/are needed to activate TSG in PDAC.

DT1 optimization improves its therapeutic efficacy. Specifically, DT-UHRF1 physical interactions, which are evaluated by target engagement studies, characterize their selectivity and physical and chemical properties. Structure activity relationships (SAR) improve properties and enhance DT1 efficacy and selectivity. As shown in the below examples, UHRF1 inactivation by short hairpin RNA (shRNA) decreased DNA methylation and suppressed PDAC proliferation. DT1 at 0.5-2 μM decreased PDAC proliferation and DNA methylation, upregulated pancreas differentiation factors, and induced differentiation-based morphological changes. In vitro and in vivo absorption, distribution, metabolism, and excretion (ADME) studies will establish the profile of DT1 therapy.

Efficacy of differentiation therapy by synergy of DT1, DAC, and THU combination therapy is evaluated in PDAC transgenic mouse models expressing oncogenic KRAS and mutant TP53 (KPC), with spontaneous and metastatic PDAC onset at 8 weeks and high mortality at 3 months. Currently, THU treatment enhances DAC distribution in the pancreas leading to low dose 0.2 mg/kg DAC treatment that produces durable therapeutic effects. Adding DT1 to this therapy will substantially decrease PDAC growth by non-cytotoxic epigenetic and cell differentiation-based methods that activate epigenetically silenced tumor suppressor genes.

PDAC is poorly differentiated and the methods by which loss of differentiation occurs are unknown. Tumor suppressor genes (TSG) are vital regulators of cell growth and division. Their expression in normal cells enhances cellular and homeostasis gene functions, so their activation could decrease aberrant tumor growth.

While not wishing to be bound by any particular theory, the inventors of the present disclosure hypothesized that mediators of epigenetic modifications may contribute to inactivation of differentiation-based tumor suppressor genes (TSG) of the pancreas to impair PDAC cell cycle exit by differentiation. The inventors of the present disclosure analyzed gene expressions of various epigenetic modifier enzymes and their effects on PDAC survival. The inventors of the present disclosure identified upregulations of Ubiquitin like PHD and Ring Finger Domain 1 (UHRF1) that strongly predicted poor PDAC survival (p<0.0009, n=182) using data mined from the cancer genome atlas (TCGA). Upregulation of UHRF1 was found in the most aggressive PDAC with KRAS and TP53 mutations, and its upregulation was found to decrease pancreas specific TSG that enhanced pancreas cell cycle exit by differentiation. Moreover, these TSG were highly methylated at their CpG islands in PDAC compared to normal pancreas. UHRF1 is known to cooperate with DNMT1 to epigenetically suppress genes through CpG gene methylations. Inactivation of UHRF1 by shRNA gene knockdown decreased global CpG methylation in PDAC cell lines and suppressed cell proliferation. Moreover, small molecule compounds NSC232003 (DT1) targeting UHRF1 and decitabine (DAC) targeting DNMT1 were found to synergistically suppress PDAC growth. Combination of DT1 and DAC treatment decreased DNA methylation, activated pancreas differentiation TSG, and induced differentiation cell cycle exit. Moreover, retinoic acid receptor corepressor (RAR-CoR) TBL1XR1 was found upregulated in PDAC and cases with high expression had poor survival. The small molecule all-trans-retinoic acid (ATRA) is known to induce differentiation by disrupting RAR-CoR such as TBL1XR1. The inventors of the present disclosure found that ATRA plus DAC increased differentiation cell cycle exits in PDAC cells and potently suppressed proliferation. In orthotopic mouse models containing PDAC xenografts, treatment with 0.2 mg/kg of DAC was limited in efficacy and did not affect tumor growth kinetics. However, treatment with THU (10 mg/kg) plus 0.1 mg/kg of DAC potently inactivated tumor growth in-vivo. The inventors of the present disclosure conclude that disruption of active corepressors could be a novel mechanism for the treatment of PDAC.

### Materials and Methods

TCGA Analysis of Pancreatic Cancer Datasets

Normalized RNA seq (RSEM) reads and raw count data were downloaded from the cancer genome atlas (TCGA http://cancergenome.nih.gov/). Expression data were analyzed using Prism software version 8 and heatmaps were generated using Morpheus gene Marker Selection (Broad Institute MIT) function using 1000 permutation test and FDR correction. TCGA next generation DNA-seq data were analyzed in cBioPortal (https://www.cbioportal.org/) and Xena Browser platforms to identify genetic variation of single nucleotide gene mutations and copy number variants across multiple gene pools. For analysis in cBioPortal, the inventors of the present disclosure validated data from two independent datasets, including a TCGA provisional dataset and a UTSW dataset that is micro-dissected using laser capture to enhance specificity of genetic variant analysis to tumor DNA. For PDAC data available through Xena Browser, the inventors of the present disclosure analyzed TCGA-hub PDAC-PAAD dataset available through (http://xena.ucsc.edu).

RNA Sequencing and Microarray Gene Expression Analysis

Microarray data with accession number GSE28735 that was generated using RNA isolated from PDAC samples (n=45) and adjacent normal pancreas (n=45) of the same patients was downloaded from the Gene Expression Omnibus (GEO) database (https://www.ncbi.nlm.nih.gov/geo/). GenePattern and Gene Marker Selection were used with a 10,000 permutations test and FDR correction to identify differentially expressed genes. Identification of gene pathways were analyzed using Reactome pathway analysis tool (https://reactome.org/PathwayBrowser/). Heat maps were generated using GenePattern suit Morpheus.

Gene Expression by Real Time qPCR

Gene expression analysis by real time quantitative polymerase chain reaction (qRT-PCR) was performed using standard methods. Gene expression of target genes was performed using either SYBR green methods or Power Green ROX dye master mix (Applied Biosystems, Foster city CA, USA Lot number 1610538). Assays were performed with an Applied Biosytems ViiA7 (Life Technologies).

DNA Methylation Analysis

DNA Methylation of normal pancreas and PDAC tumor samples was analyzed using data available through the MethHC database (http://methhc.mbc.nctu.edu.tw/php/index.php) using the PAAD dataset. Methylation analysis was performed at transcriptional start sites (TSS), promoters, and CpG island. For global DNA methylation changes, cells were transfected with shRNA against human UHRF1 (Dharmacon Horizon, catalog V3SH7590-225743149) for 96 hours or treated with THU (1 μM) DT1 (0.5-15 μM) or 5-aza-cytidine (1 μM). DNA was isolated using the Clonetech NucleoSpin DNA rapid lyse kit (catalog 740110.1) following manufacture's recommended conditions. 100 ng of isolated DNA was used to quantify DNA methylation using the 5mC-Long Interspersed Nucleotide Element 1 (LINE-1) kit from Active Motif (catalog number 55017) following the manufacture's recommendations. A Colorimetric ELISA readout was measured on a BioTek synhergy HI reader at 450 nm.

Western Blot Analysis

Western blot was on a LICOR instrument following the manufacturer's standard protocol

Pancreatic Cancer Cell Lines

A panel of pancreatic cancer cell lines (PCCs) was ordered from ATCC (https://www.atcc.org). Cells were cultured in RPMI media supplemented with 10% FBS and 1% penicillin streptomycin (100 units/ml of penicillin and 100 mg/mL of streptomycin) in a 37° C. incubator with 5% Carbon dioxide (CO₂). All cell lines were confirmed to be mycoplasma-free using a Mycoalert detection kit (Lonza Allendale N.J., catalog #LT07-218) as per manufacturer's protocol. For DT1, DAC, and THU treatments, 1000 cells were plated in a 96 well plate overnight. Cells were treated three times at 24, 48, and 72 hours post-plating using concentrations of 0.2-2 μM of DT1 and DAC and 1-2 μM of THU independently or in combination. DMSO was used as a vehicle control. Cell proliferation was measured in a 37° C., Incucyte incubator (IncuCyte™ live-cell, Essen biosciences, Michigan USA) with 5% CO₂ for automated live cell proliferation in real time, and images were taken every 6 hours for up to 160 hours. Cells were also counted on a Beckman coulter automated cell counter.

Orthotopic Mouse Models of Pancreatic Cancer

Orthotopic mouse models with human PDAC cells were generated using NOD/SCID mice. In vivo BLI was conducted on a NiteOwl system and acquisition software. Data were analyzed using eLumenate analysis software. For the primary tumor, signal intensity was quantified as the sum of all detected photon counts within a region of interest selected by an automated maximum entropy method and normalized to the area of that region of interest. For the metastatic region, a region of interest was drawn manually in a “crescent” shape which encompassed all of the abdominal region except the primary tumor and maintained a boundary at least 2 mm away from the ROI for the primary. The signal in the metastatic region was again quantified by dividing the sum of all detected photon counts in the region by the area of the region.

Statistical Analysis

Results were expressed as the mean SEM where applicable. P-values were calculated by two-tailed, unpaired student's t-test for paired comparisons using GraphPad Prism version 8. For differential gene expression, Gene Marker Selection was used for FDR correction and results were represented using a 10000 permutation test. P-value ≤0.05 was considered significant.

Survival Analysis

Survival analysis for patients with high versus low corepressors (e.g., DNMT1, UHRF1 or TBL1XR1) levels was analyzed using a Xenabrowser platform.

### Results

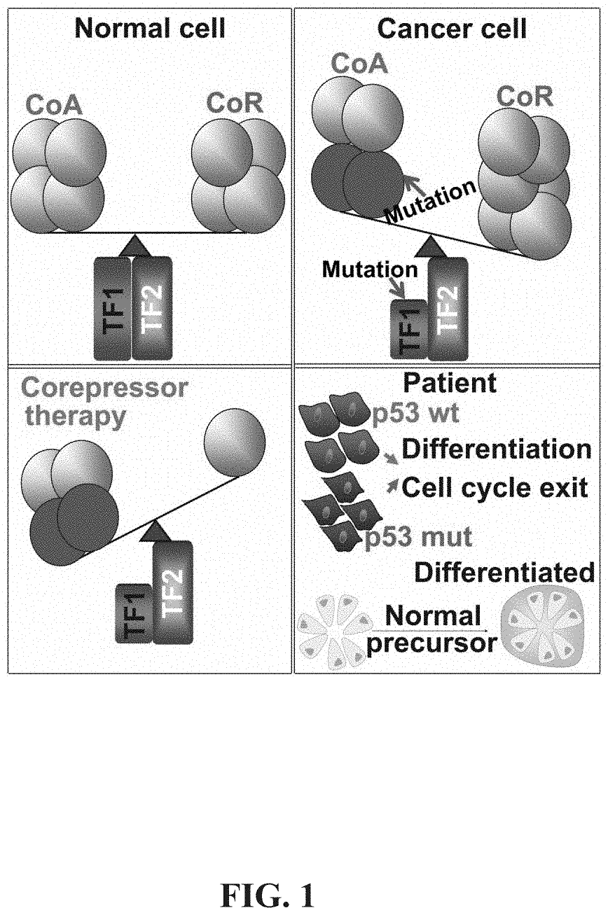

Referring now to FIG. 1, the inventors of the present disclosure found that corepressor epigenetic enzymes are upregulated in cancer cells and altered the expression of differentiation factors (Enane et al 2017, Enane et al 2018). This led to the hypothesis that corepressor disruption is a novel mechanism for cancer therapy. The hypothesis, which is derived from a series of previous research of the inventors of the present disclosure, suggested that pancreas lineage transcription factors (TF1, TF2, e.g., PDX1 and FOXA2) coordinate to recruit coactivator enzymes (CoA e.g., ARID1A) or corepressor enzymes (CoR, e.g., DNMT1) to turn on or turn off genes, respectively. This process is balanced in normal cells. However, in malignant cells such as pancreatic cancer cells, genetic variation (red arrow) produces favorable recruitment of CoR off enzymes to TF1, TF2 inactivating differentiation tumor suppressor genes. Inhibition of active corepressors could be a basis for corepressor therapy that rebalances this interaction to CoA to reactivate suppressed tumor suppressor genes and turn differentiation cell cycle exit. This approach may terminate proliferation in a tumor regardless of mutation cell death promoting genes such as TP53 (gene for p53 protein).

Referring now to FIG. 2, Ubiquitin like PHD and Ring Finger Domain 1 (UHRF1) and DNA Methyl-transferase-1 (DNMT1) cooperate to epigenetically silence differentiation genes. In particular, UHRF1 cooperation with DNMT1 results in epigenetic repression of TSG. Disruption of the SET and RING associated domain (SRA) of UHRF1 by small molecule NSC232003 (DT1) and degradation of DNMT1 using small molecule decitabine (DAC) reactivates epigenetically silenced TSG. Reactivation of differentiation-based TSG in pancreatic cancer cells induces cell cycle exit by differentiation.

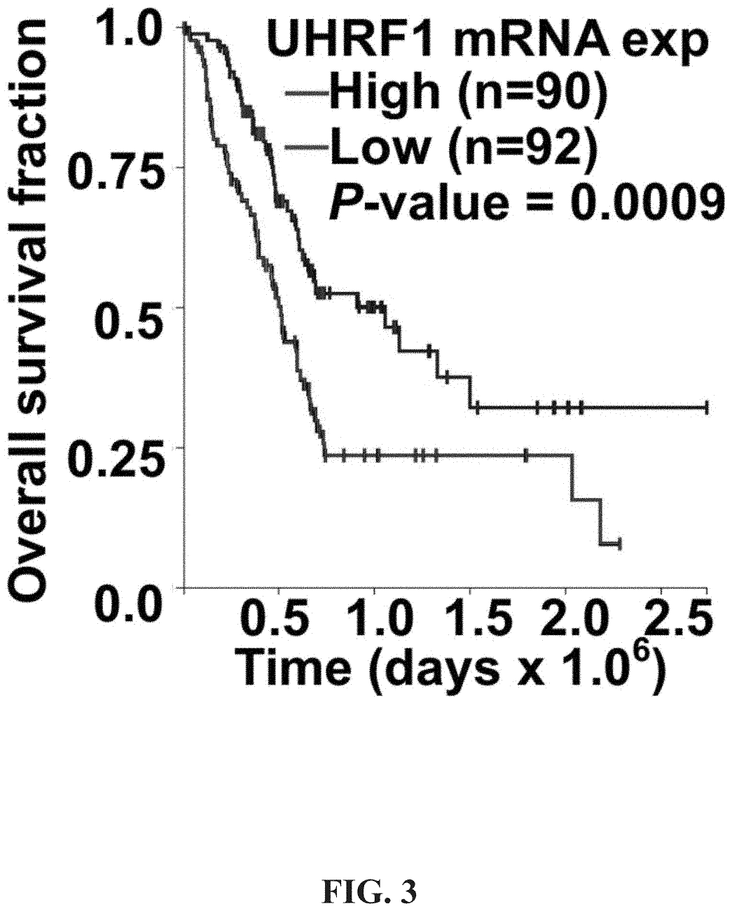

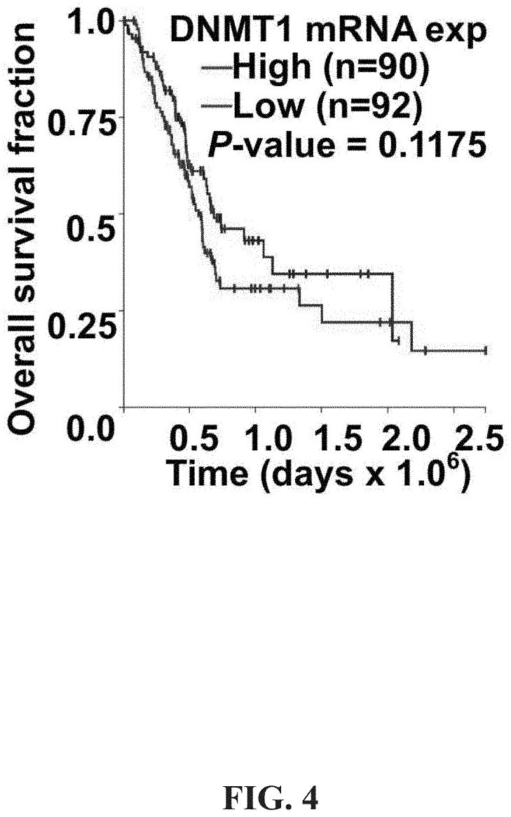

Referring now to FIG. 3, analysis of corepressor UHRF1 gene expression in PDAC patient samples was performed using data from TCGA through Xena Browser platform. Cases were sorted by high (n=90) versus low (n=92) UHRF1 levels and survival analysis was performed. LogRank p=0.0009. Referring now to FIG. 4, analysis of corepressor DNMT1 gene expression in PDAC patient samples was performed using RNAseq data from TCGA through Xena Browser platform. Cases were sorted by high (n=90) versus low (n=92) DNMT1 levels and survival analysis was performed. LogRank p=0.1175. Referring now to both FIG. 3 and FIG. 4, upregulation of UHRF1 predicted poor PDAC patient survival (p-value=0.0009) and although p-value was not significant (p=0.1175), PDAC cases with high DNMT1 also had poor survival.

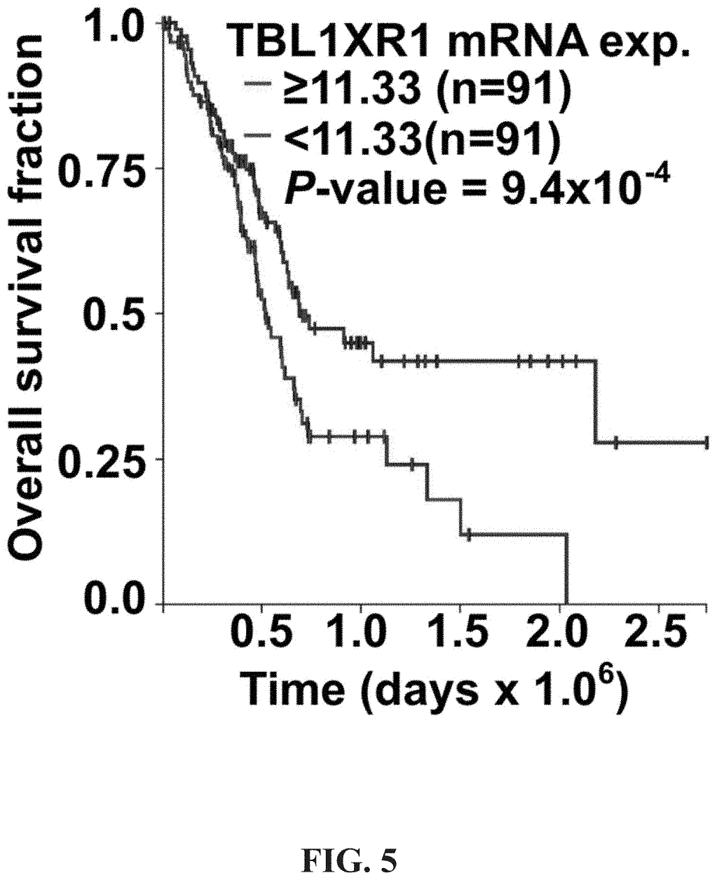

Referring now to FIG. 5, analysis of corepressor TBL1XR1 gene expression in PDAC patient samples was performed using RNAseq data from TCGA through Xena Browser platform. Cases were sorted by high (n=91) versus low (n=91) TBL1XR1 levels and survival analysis was performed. LogRank p=9.4×10⁴. Retinoic acid receptor corepressor (RAR-CoR) TBL1XR1 upregulation also predicted poor PDAC survival.

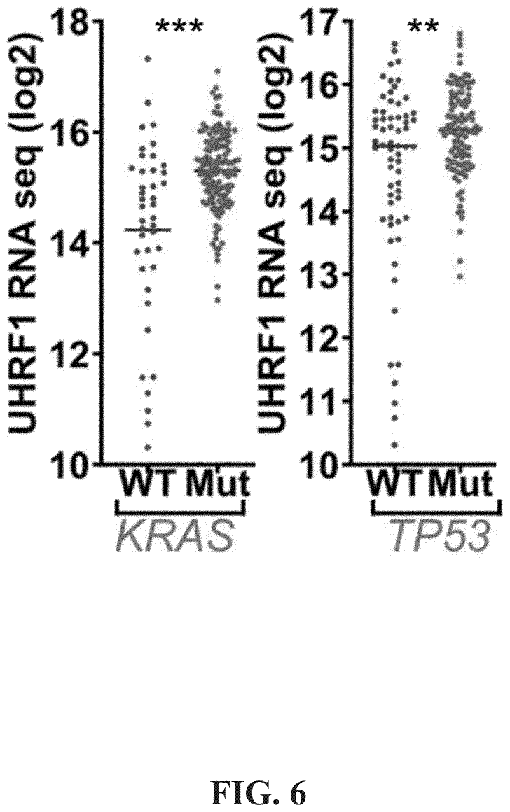

Referring now to FIG. 6, cases containing KRAS and TP53 mutational status were downloaded from TCGA sorted by UHRF1 expression levels (RNAseq) through Xena browser. UHRF1 expression levels were compared between cases with wild-type KRAS or TP53. *** Wilcoxon p-value <0.0001, ** Wilcoxon pvalue <0.001. Data was analyzed using GRAPHPAD PRSIM v8. High UHRF1 expression was found in the aggressive PDAC with KRAS and TP53 gene mutations.

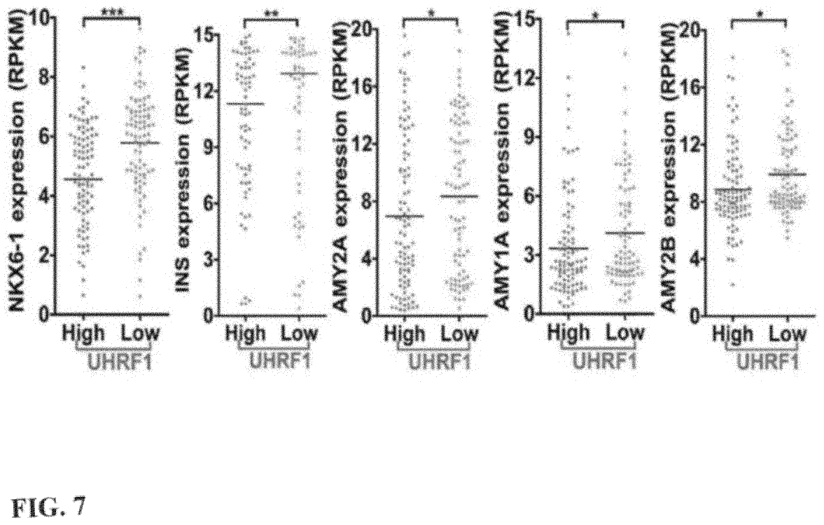

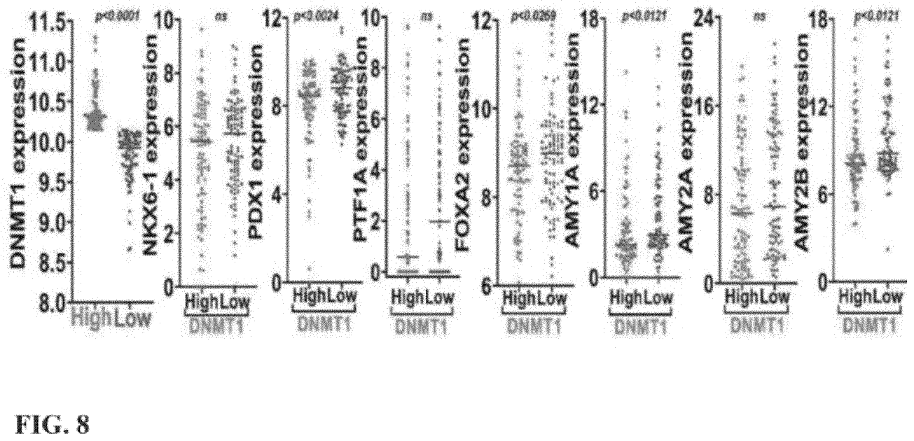

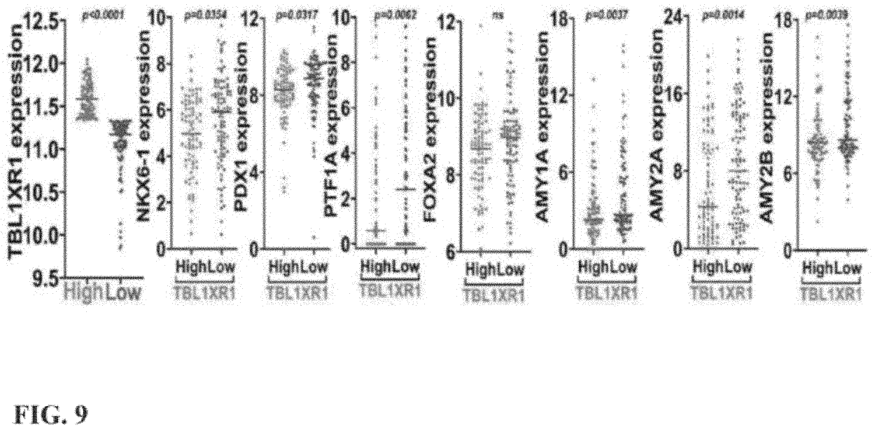

Referring now to FIG. 7, expression of pancreas terminal differentiation and homeostasis factors (NKX6-1, INS, AMY2A, AMY1A and AMY2A) were analyzed using TCGA RNAseq data in UHRF1 high (blue dots) versus UHRF1 low (red dots) cases. *** Wilcoxon p-value <0.0001, ** Wilcoxon p-value <0.001, * Wilcoxon p-value <0.05. Data was analyzed using GRAPHPAD PRSIM v8. Referring now to FIG. 8, pancreas terminal differentiation markers and homeostasis genes were analyzed in PDAC expressing high DNMT1 (red dots) versus with PDAC with low DNMT1 levels (blue dots). TCGA PAAD datasets, p-value=Wilcoxon. Referring now to FIG. 9, pancreas terminal differentiation markers and homeostasis genes were analyzed in PDAC expressing high TBL1XR1 (red dots) versus with PDAC with low TBL1XR1 levels (blue dots). TCGA PAAD datasets, p-value=Wilcoxon. Referring now to FIGS. 7-9, pancreas differentiation markers and tumor suppressor genes/TSG (e.g., NKX6-1, AMY2A/B) were downregulated in high UHRF1, DNMT1, and TBL1XR1-expressing PDAC.

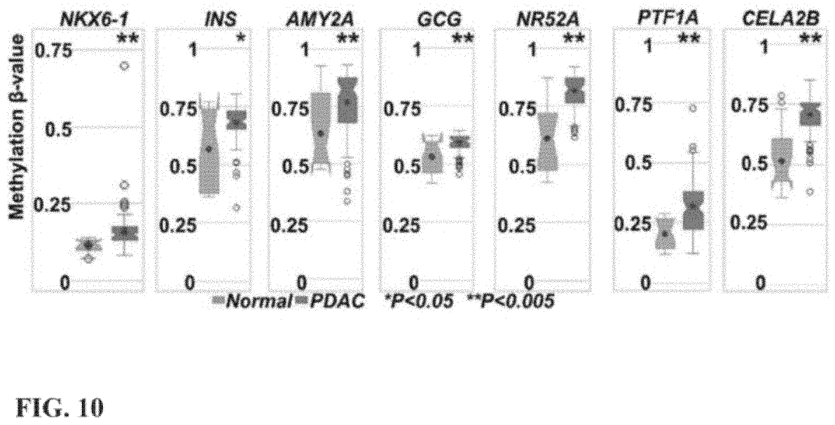

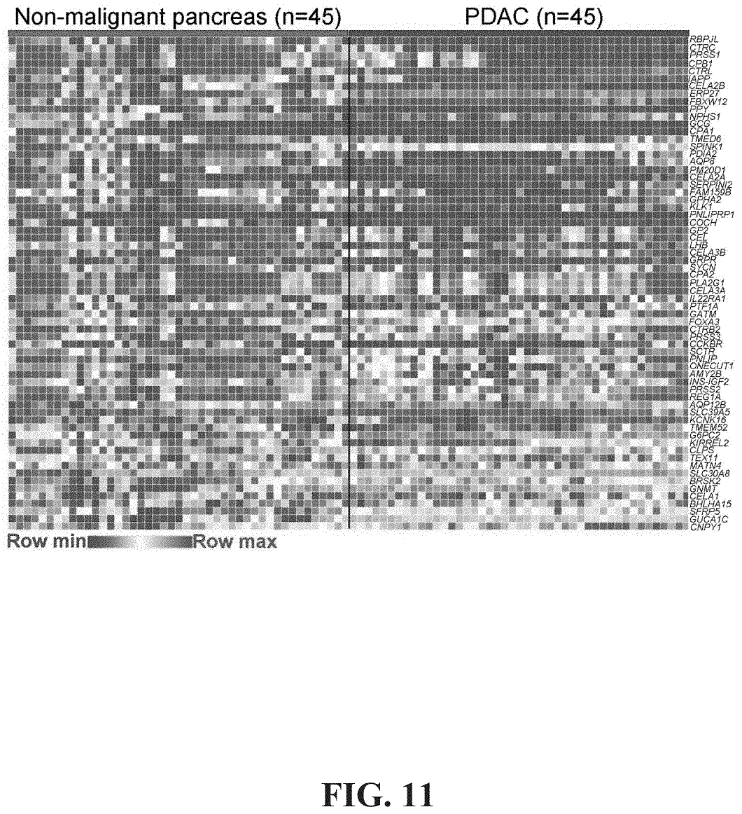

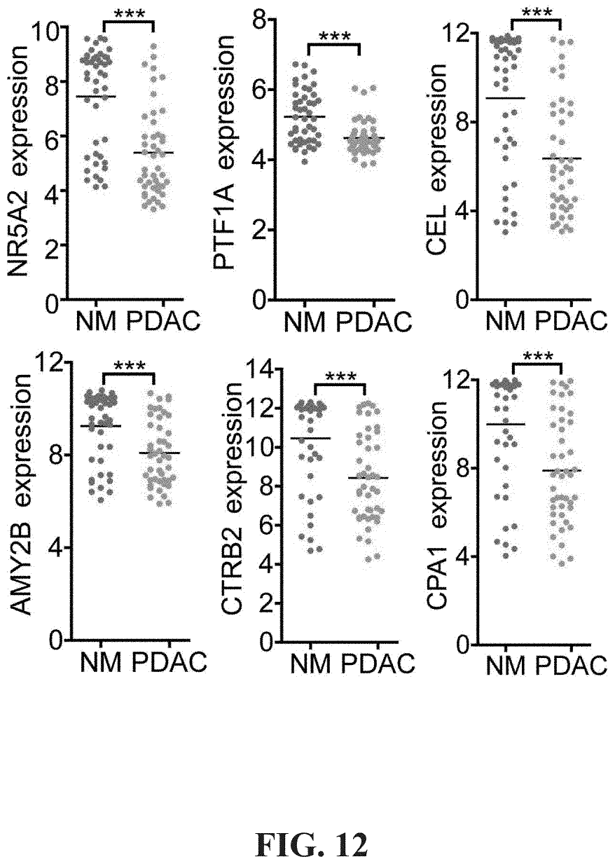

Referring now to FIG. 10, differentially methylated genes were downloaded from the database of DNA Methylation and gene expression in Human Cancer (MethHC). CpG Methylation of pancreas specific genes (NKX6-1, INS, AMY2A, GCG, NR5A, PTF1A, and CELA2B) in normal versus PDAC samples is shown. ** Wilcoxon pvalue <0.005, * Wilcoxon pvalue <0.05. These TSG were also found to have high DNA methylation at their CpG islands when comparing methylation of normal versus PDAC tumor DNA. Referring now to FIG. 11, microarray data with accession number GSE28735 was downloaded from GEO database. Differentially expressed genes (DEG) were generated using broad institute Morpheus gene through Genepattern suite using marker selection function with 1,000 permutation test. Pathway analysis of the DEG was generated using reactome and DAVID gene ontology pathway analysis tools to identify pancreas specific markers. Referring now to FIG. 12, microarray data with accession number GSE28735 was downloaded from GEO database. Expression levels of pancreas specific markers were analyzed using GRAPHPAD PRISM version 8. ** Wilcoxon pvalue<0.0001. Referring now to FIGS. 10-12, although differentiation-based TSG are not mutated in primary PDAC, they were found to be methylated and epigenetically silenced and had decreased expression in PDAC samples.

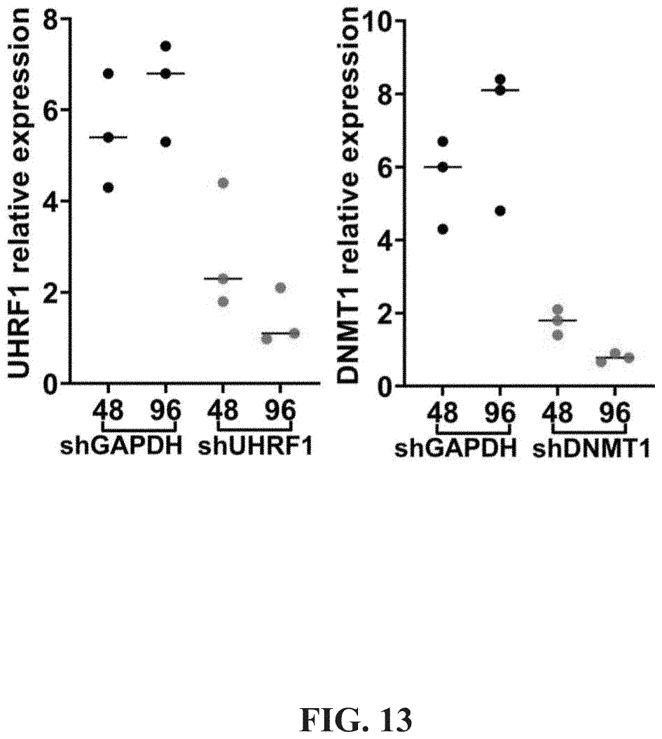

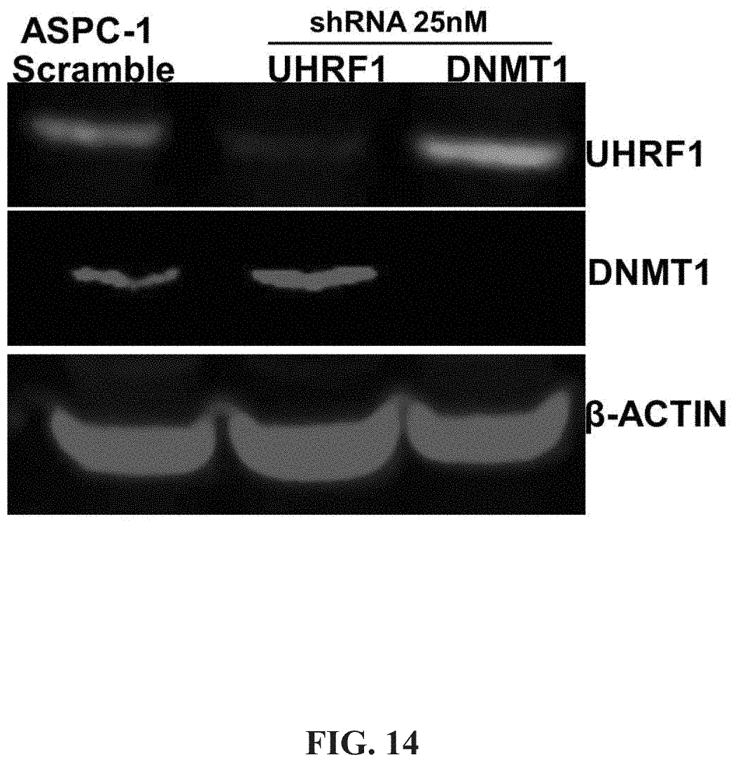

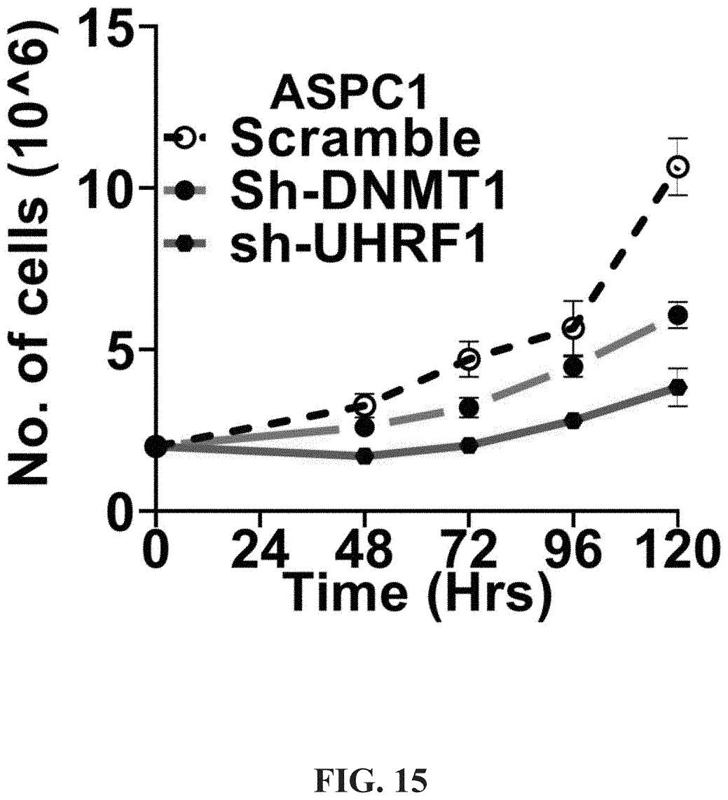

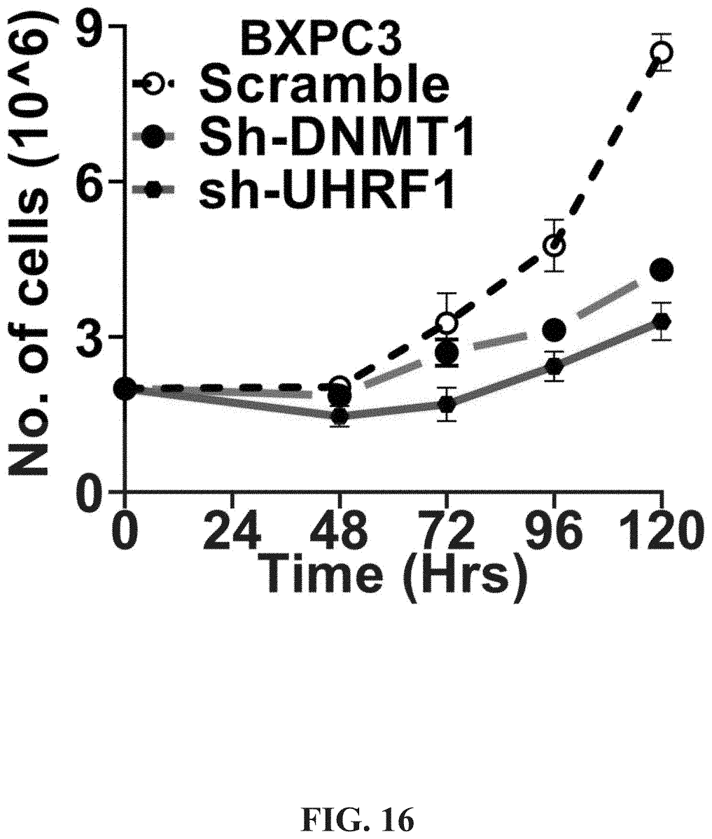

Referring back to FIG. 2, UHRF1 is known to recruit DNMT1 to enhance maintenance methylation at hemi-methylated DNA, which is a repressive epigenetic event that silences TSG7-9. The DNA binding SRA domain of UHRF1 recruits DNMT1 to drive DNA methylation. UHRF1 and DNMT1 have been linked to the epigenetic suppression differentiation factors in the treatment of recalcitrant solid tumors. Thus, anti-corepressor therapy disrupting DNMT1 and UHRF1 can reactivate epigenetically silenced differentiation genes. Referring now to FIG. 13, pancreatic cancer cell line ASPC1 was transfected with shRNA against DNMT1 and UHRF1. shGAPDH was used as non-target control. RNA was isolated at 48 and 96 hours and used to quantify expression levels of DNMT1 and UHRF1 using gene expression qPCR analysis. Referring now to FIG. 14, ASPC1 cells were treated with 25 nM of sh-UHRF1 and sh-DNMT1 and monitored for 96 hours. At 96 hours, protein was isolated and the levels were analyzed on a LICOR instrument using antibodies against UHRF1 and DNMT1. Non-targeting sh-scramble was used as the control. Referring now to FIG. 15, pancreatic cancer cell line ASPC1 was transfected with shRNA against DNMT1 and UHRF1. Cell proliferation was monitored for 120 hours. Cells were counted on an automated cell counter at 48, 72, 96, and 120 hours post transfection. Referring now to FIG. 16, pancreatic cancer cell line BXPC3 was transfected with shRNA against DNMT1 and UHRF1. Cell proliferation was monitored for 120 hours. Cells were counted on an automated cell counter at 48, 72, 96, and 120 hours post transfection. Referring to FIGS. 13-16, using sh-RNA gene silencing studies, UHRF1 and DNMT1 inactivation decreased aberrant PDAC cell proliferation. The strongest suppression was seen in sh-UHRF1 transfected cells.

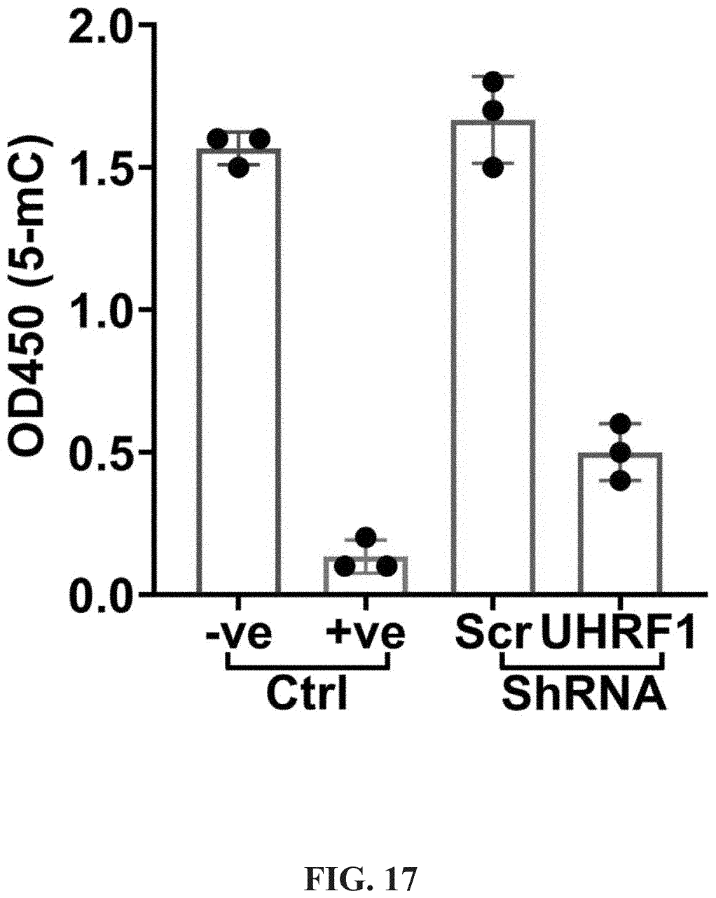

Referring now to FIG. 17, pancreatic cancer cell line ASPC1 was transfected with shRNA against UHRF1. DNA (100 ng) was isolated from shUFRF1 and scramble transfected cells at 96 hours post transfection and was used for quantification of global 5-methyl-cytosine methylation using LINE1 global DNA methylation kit (ACTIVE MOTIF). UHRF1 inactivation also decreased global DNA methylation in PDAC cells analyzed by LINE1 global DNA methylation assay.

Referring now to FIGS. 18-22, the inventors of the present disclosure validated small molecule compounds, including NSC232003 (DT1) that targets UHRF111, NSC127716 (Decitabine or DAC) that degrades DNMT112, NCSC112907 (Tetrahydrouridine or THU) that inhibits CDA13, and NSC122758 (ATRA) that targets retinoic acid receptor pathway14.

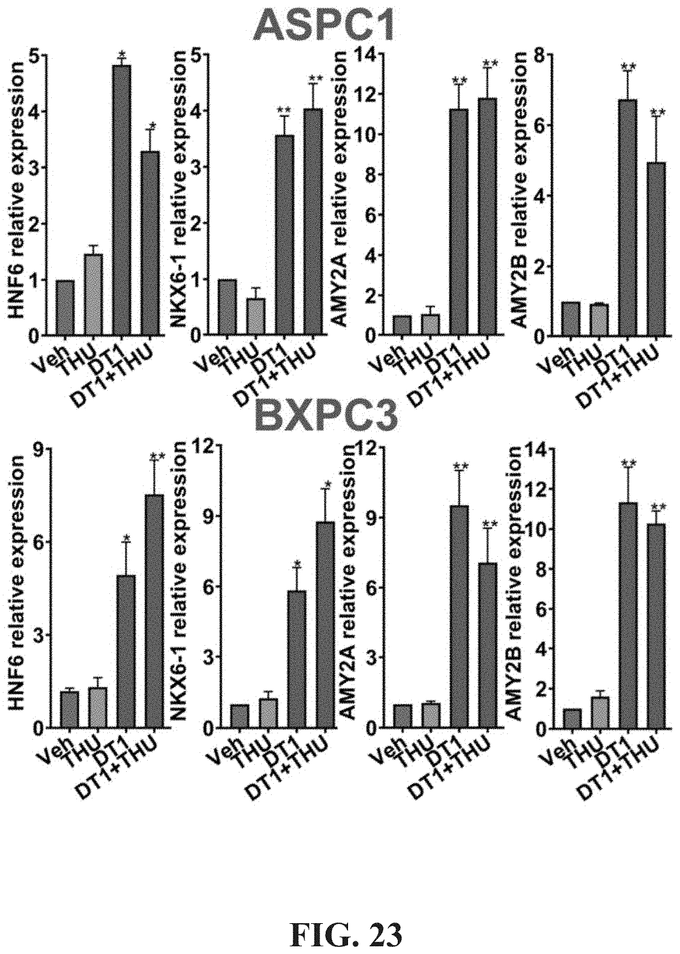

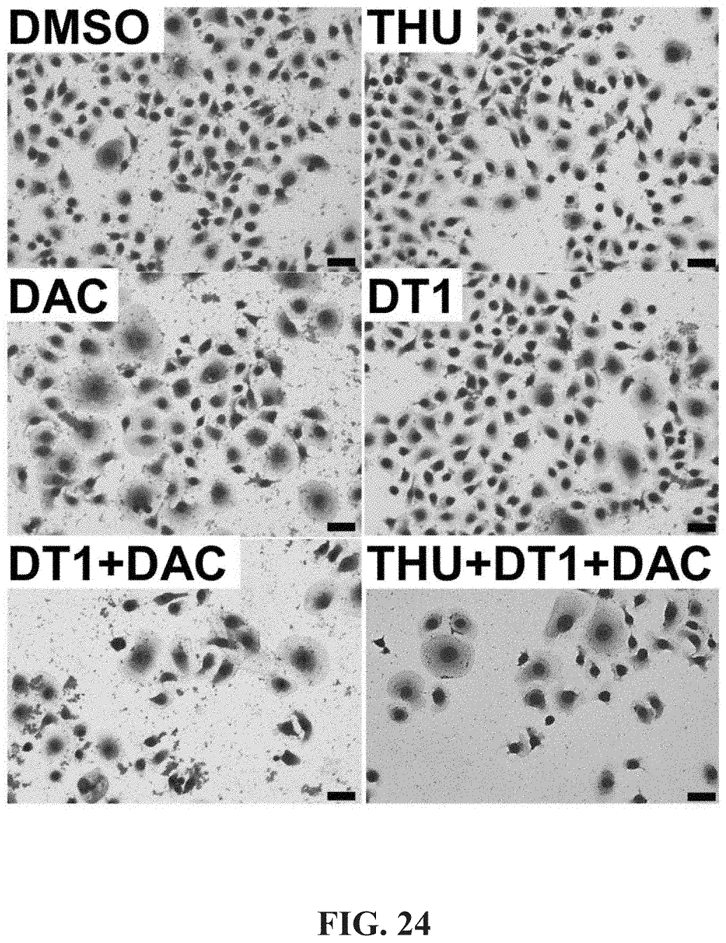

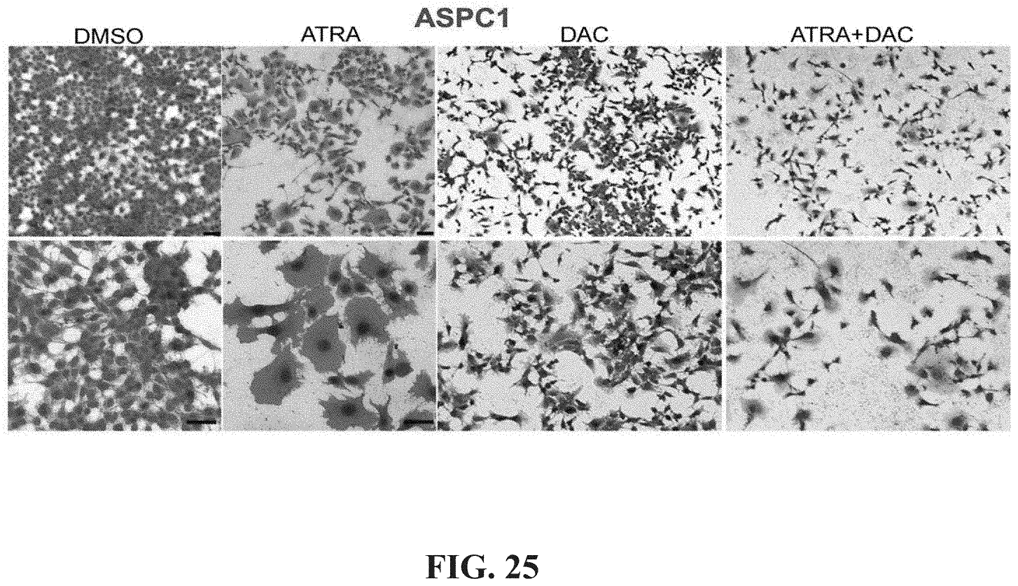

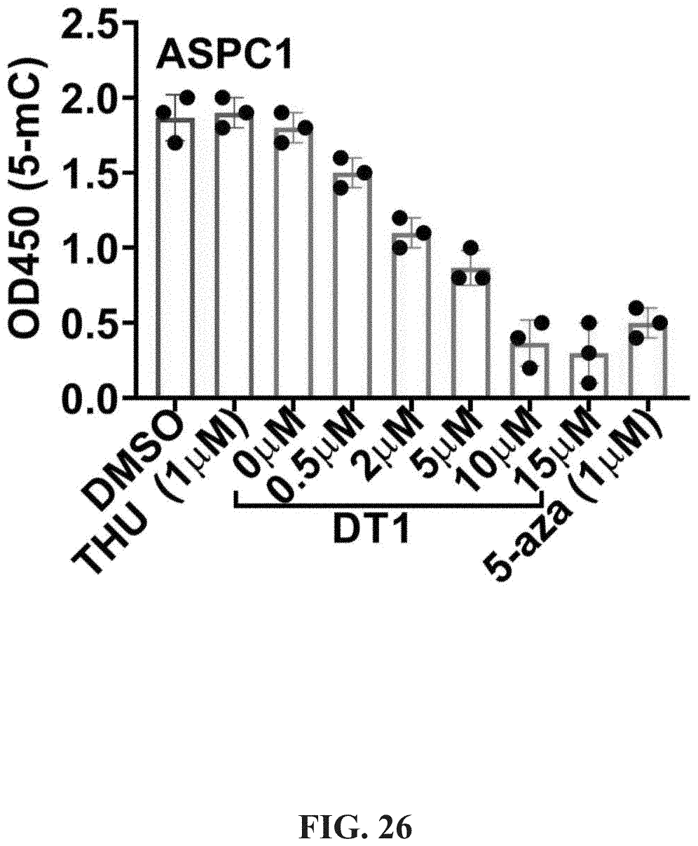

Referring now to FIG. 23, primary PDAC cells were incubated with 2 μM of DT1 and 1 uM of THU for 96 hrs. RNA was isolated and gene expression pancreas differentiation factors were analyzed in ASPC1 and BXPC3 cells. Referring now to FIG. 24, primary PDAC cell line ASPC1 was treated with 1 μM of THU, 2 μM of DT1, and 1 μM of DAC, or the combination of 0.5 μM of DT1 plus 0.2 μM of DAC, or 1 μM of THU and 0.5 μM of DT1 plus 0.2 μM of DAC. Morphological analysis was performed using a giemsa staining method to determine induction of differentiation (enlargement of cytoplasm and reduction of nuclear compartments). Black bar=200× magnification. Referring now to FIG. 25, primary PDAC cell line ASPC1 was treated with 100 nM of ATRA and 1 μM of DAC, or combination of 100 nM of ATRA plus 200 nM of DAC. Morphological analysis was performed using a giemsa staining method to determine induction of differentiation (enlargement of cytoplasm and reduction of nuclear compartments). Black bar=200× magnification. Referring now to FIG. 26, pancreatic cancer cell line ASPC1 was treated with DMSO as control, 1 μM of THU, and varying DT1 concentrations. DNA (100 ng) was isolated at 96 hours post transfection and was used to quantify global 5-methyla-cytosine (5-mC) methylation using LINE1 global DNA methylation kit (ACTIVE MOTIF). DNA from cells treated with 1 μM of 5-aza-cytidine (5-aza) was used as a positive control. Referring now to FIGS. 23-26, DT1 and DAC decreased cell growth through modification of DNA methylation, upregulated epigenetically silenced TSG, and induced cell morphological changes (increased cytoplasm and decreased nucleus) consistent with epithelial differentiation. As nucleoside analogs, DT1 and DAC efficacy was limited by high CDA expression in the pancreas. The inventors of the present disclosure inhibited CDA by THU (1 μM) followed by non-cytotoxic DT1 and DAC treatment (0.2-0.5 μM). This treatment further decreased PDAC proliferation, increased expression of pancreas differentiation genes, and decreased global DNA methylation.

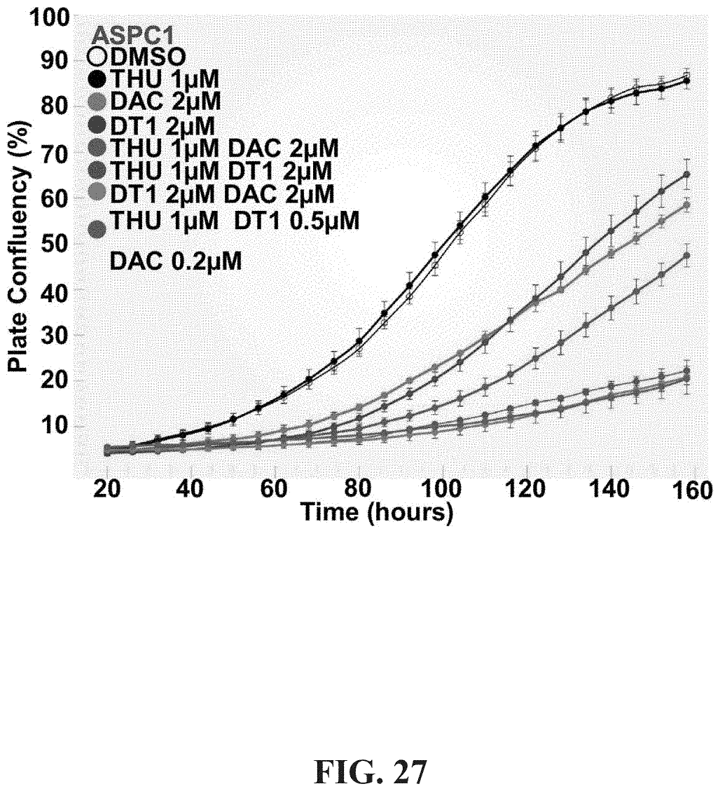

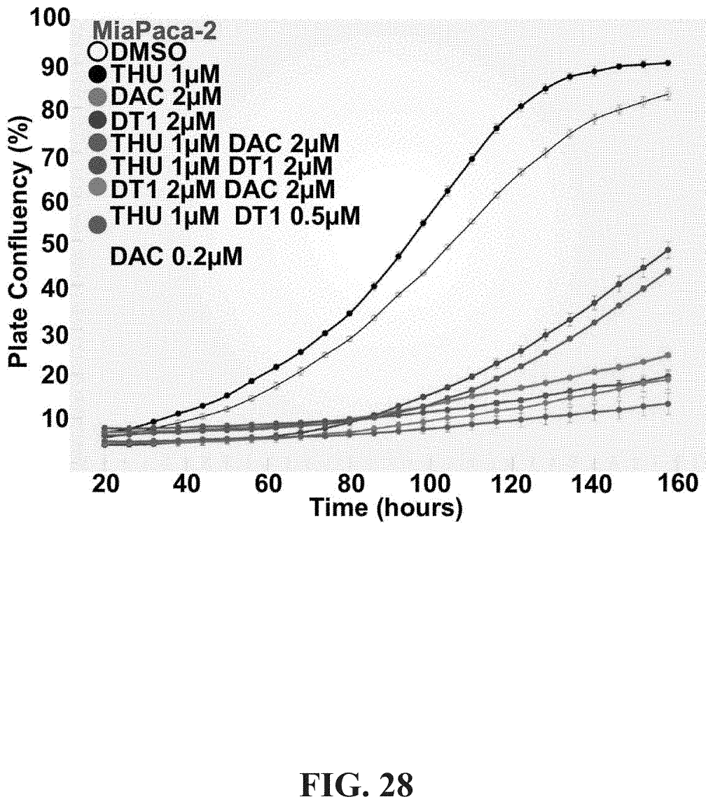

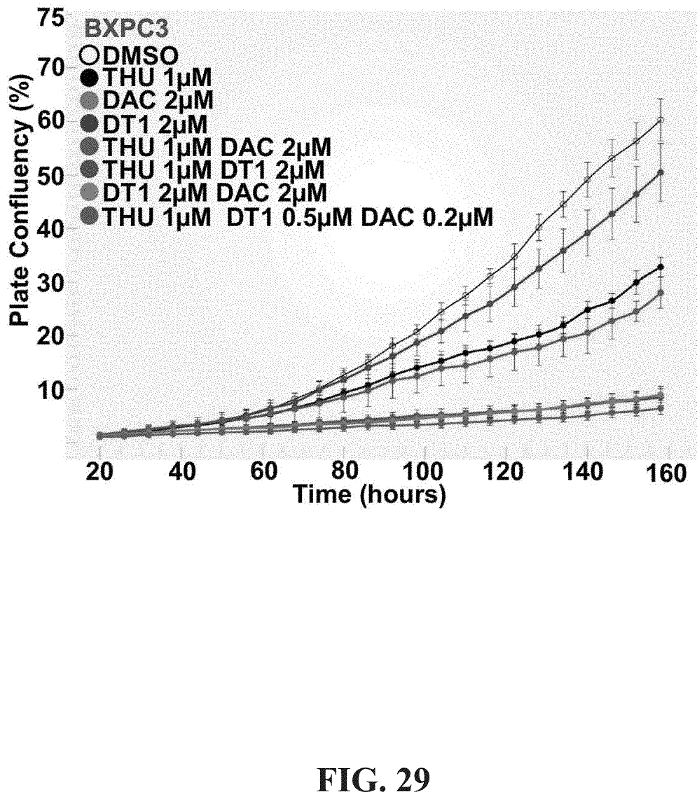

Referring now to FIG. 27, primary PDAC cell line ASPC1 was incubated with 2 μM DT1 to inhibit UHRF1 and/or DNMT1 inhibitor DAC or CDA inhibitor THU (1 μM) or 0.5 M DT1, 0.2 μM DAC and THU (1 μM). Cell proliferation was quantified by automated IncuCyte incubator for 160 hours. FIG. 27 shows proliferation of ASPC1 cell line in drug treated versus control. Referring now to FIG. 28, primary PDAC cell line MiaPaCa-2 was incubated with 2 μM DT1 to inhibit UHRF1 and/or DNMT1 inhibitor DAC or CDA inhibitor THU (1 μM) or 0.5 M DT1, 0.2 μM DAC, and THU (1 μM). Cell proliferation was quantified by automated IncuCyte incubator for 160 hours. FIG. 28 shows proliferation of MiaPaCa-2 cell line in drug treated versus control. Referring now to FIG. 29, primary PDAC cell line BXPC3 was incubated with 2 μM DT1 to inhibit UHRF1 and/or DNMT1 inhibitor DAC or CDA inhibitor THU (1 μM) or 0.5 M DT1, 0.2 μM DAC, and THU (1 μM). Cell proliferation was quantified by automated IncuCyte incubator for 160 hours. FIG. 29 shows proliferation of BXPC3 cell line in drug treated versus control. Decitabine (DAC) is a clinically available drug that degrades DNMT1 to decrease DNA methylation while DT1 targets UHRF1. Referring to FIGS. 24 and 27-29, the inventors of the present disclosure evaluated DT1 and DAC efficacy in PDAC and found a potent synergistic effect that decreased PDAC growth and induced differentiation-based morphological changes. These synergistic effects were most potent when THU, DT1, and DAC were combined.

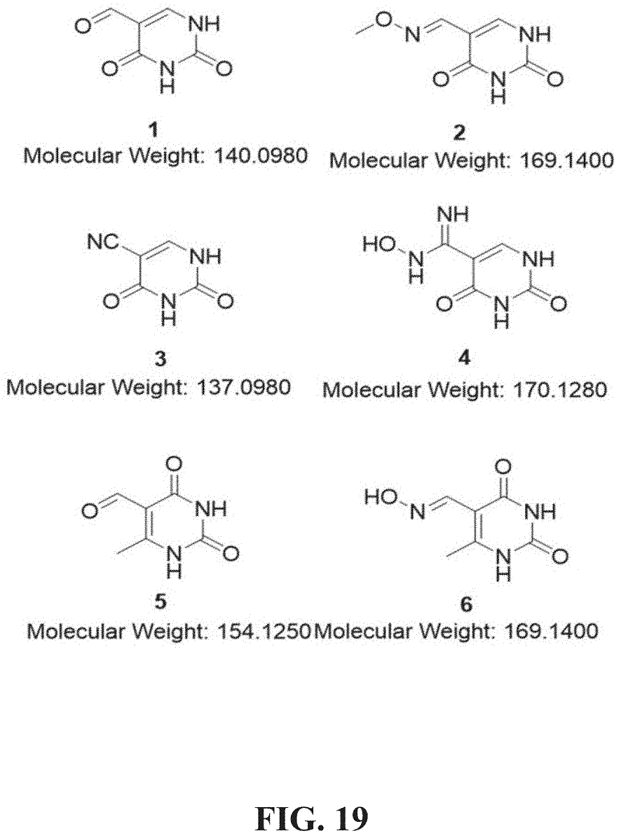

Referring now to FIG. 19, DT1 activity was observed at a micromolar range. The inventors of the present disclosure synthesized 6 novel DT1 derivatives using structure activity relationship (SAR) at the UHRF1 SRA domain that molecularly predicts higher potency that are undergoing investigation.

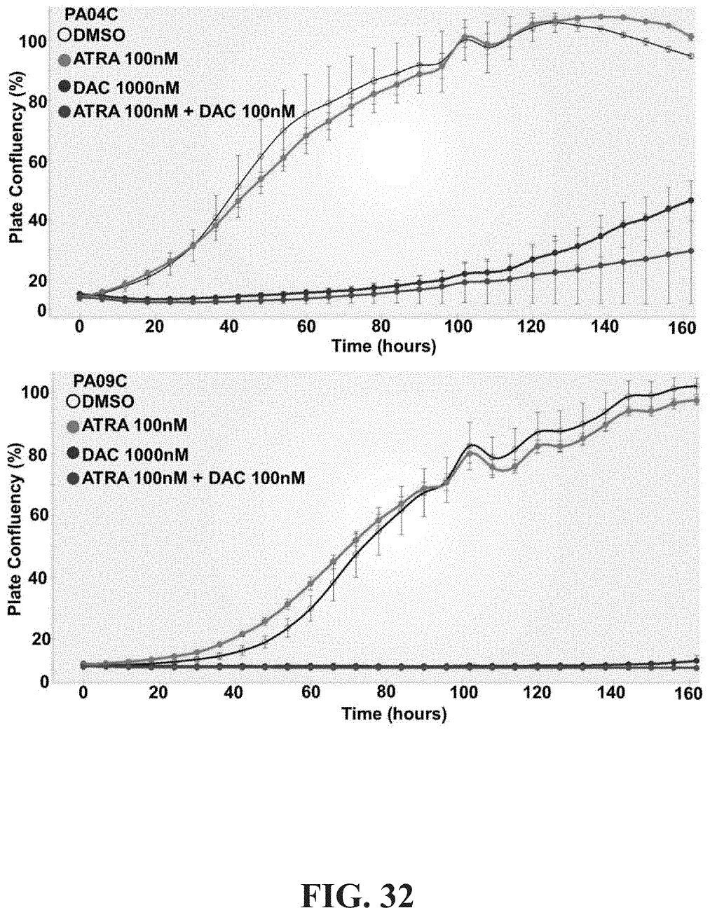

Referring to FIG. 32, patient derived cell lines PA04C (top graph) and PA09C (bottom graph) were treated with 100 nM ATRA or 1000 nM DAC versus the combination of 100 nM of ATRA+DAC. Proliferation was measured by automated IncuCyte incubator. Referring back to FIG. 25 and FIG. 32, the inventors of the present disclosure also found that the combination of DAC and ATRA induced differentiation-based morphological changes and that this combination potently suppressed patient derived pancreatic cancer cell proliferation.

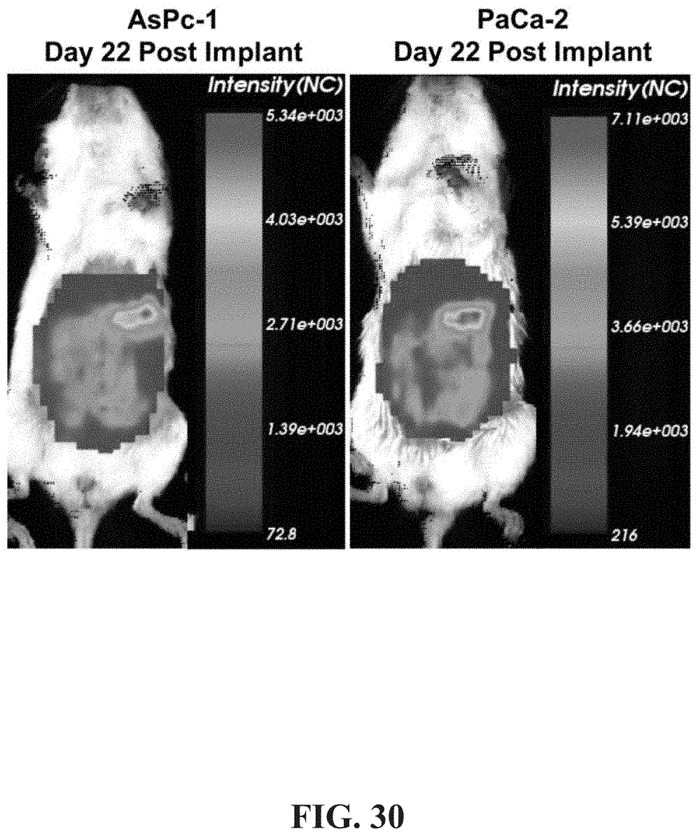

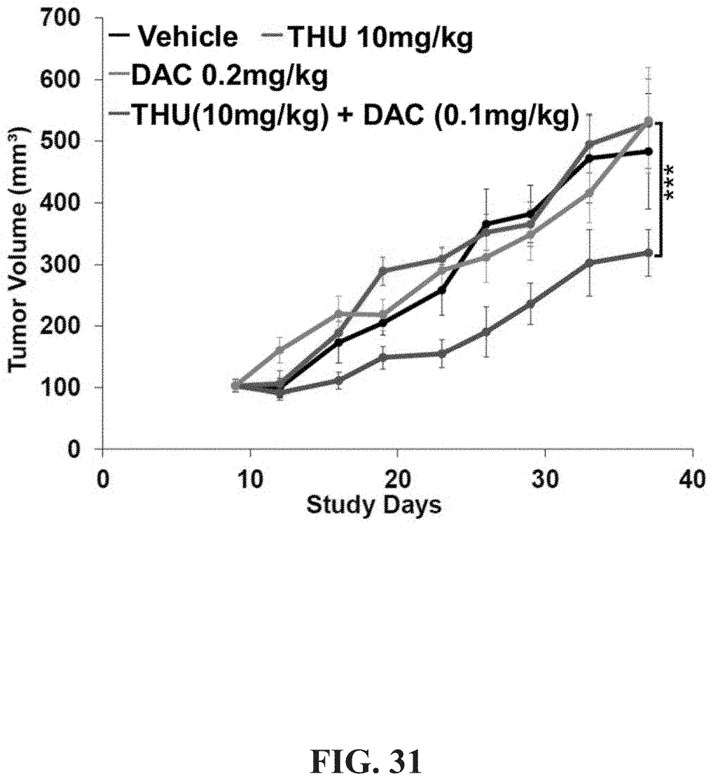

Referring to FIG. 30, bioluminescent and primary PDAC cell lines ASPC1 and MiPaCa-2 were used to generate PDAC xenografts for therapeutic validation. At 22 days post injection of tumor cells, tumor growth and metastatic dissemination are detected in this orthotopic models in NOD/SCID mice. Referring to FIG. 31, orthotopic xenografts of PDAC with AsPC-1 cells were treated with vehicle control, Tetrahydrouridine (THU, 10 mg/kg), Decitabine (DAC, 0.2 mg/kg), or combination of THU (10 mg/kg) with DAC (0.1 mg/kg) three times weekly. Tumor volume was assessed twice weekly for 35 days and the suppression of the tumor kinetics was most potent in the THU-DAC group ***p<0.0001. Referring back to FIGS. 30 and 31, in orthotopic mouse models containing PDAC xenografts, treatment with 0.2 mg/kg of DAC or 10 mg/kg of THU was limited in efficacy and did not affect tumor growth kinetics. However, as illustrated in FIG. 31, treatment with THU (10 mg/kg) plus 0.1 mg/kg of DAC potently inactivated tumor growth in-vivo. The inventors of the present disclosure conclude that combined disruption of UHRF1 using DT1 and DNMT1 using DAC, and retinoic acid receptor using ATRA is a novel method to treat pancreatic cancer by activating differentiation-based tumor suppressor genes and enhancing cell differentiation without inducing cell death.

### CONCLUSIONS

The epigenetic enzymes (e.g., UHRF1 and DNMT1) may cooperate to epigenetically silence pancreas TSG that regulate cell cycle exit by differentiation. Epigenetic gene repression also occurs via the retinoic acid receptor pathway CoR TBLXR1 activity. Pharmacologic targeting of UHRF1 (DT1) and DNMT1 (DAC) synergistically inactivate PDAC proliferation. As nucleoside analogs, DT1 (derivative of uracil) and DAC (derivative of cytidine) efficacy enhanced inhibition of CDA, which is a pyrimidine metabolic enzyme that limits efficacy of nucleoside analog-based compounds. Thus, DT1 and DAC combination therapy may be enhanced by THU treatment to treat PDAC by enhancing cell cycle exit by cell differentiation. Moreover, disruption of corepressors in the retinoic acid receptor pathway can be used independently or in combination with DAC and/or DT1 for novel non-cytotoxic therapy that induces differentiation cell cycle exit in PDAC cells. In preliminary research, the inventors of the present disclosure found that THU-DAC combination potently suppressed PDAC growth in-vivo and this treatment was not toxic.

Other conclusions may be drawn from these examples.

The small molecule DT1 targets the DNMT1 recruiting domain of UHRF1 to decrease UHRF1 mediated DNA methylation. Moreover, the epigenetic modifications of DNMT1 are pharmacologically validated for differentiation-based cancer therapy by the molecule decitabine (DAC) that is approved for treatment of MDS-leukemia.

A combinatorial epigenetic therapy using DT1-DAC epigenetic drugs that engage epigenetically silenced TSG is described. In PDAC, 79% of cases had upregulated UHRF1 and KRAS and TP53 mutations (see FIGS. 3 and 6). Mutations of TP53, KRAS, and UHRF1 overexpression are also common in multiple human malignancies. UHRF1, a multifunctional protein, contains a SET and RING (SRA) DNA binding domain that preferentially recognizes hemi-methylated CpG dinucleotides and recruits DNMT1 for maintenance of DNA methylation. This epigenetic event suppresses multiple TSG during cancer self-replication. UHRF1 degradation decreases DNA methylation and self-replication; however, no current therapies have been designed to clinically modulate its role in cancer. UHRF1 and DNMT1 oncogenic cooperation impairs cell cycle exit by differentiation, and the epigenetic component of this interaction is reversible by pharmacologic manipulation. UHRF1 upregulation predicted PDAC poor survival and decreased expression of pancreas specific differentiation factors through CpG methylation in preliminary work (see FIGS. 3, 6, and 10). UHRF1 and DNMT1 produce epigenetic modifications at pancreas gene loci that can be pharmacologically reversed to induce PDAC cell cycle exit by differentiation.

Apoptosis and differentiation tightly regulate self-replication in metazoan biology. Cell differentiation that terminates self-replication by increasing differentiation cell cycle exits, results in functional cells for homeostasis. The most aggressive malignancies are poorly differentiated by unknown mechanisms impeding development of differentiation engaging therapies. Engaging cell differentiation to decrease self-replication is non-lethal and non-cytotoxic to normal stem cells. Apoptosis therapies are thus presumed cytotoxic, whereas differentiation therapies are non-lethal to normal cells but suppress tumor cell growth. Because inducing apoptosis is cytotoxic to normal cells, yet resisted by the cancer cells, an alternative therapy engaging non-mutated but epigenetically silenced differentiation factors terminates malignant growth, independent of apoptosis. While exponential proliferation of normal precursors is terminated by cell differentiation, malignant self-replication is not succeeded by forward-differentiation due to aberrant transcriptional repressor activities. Druggable repressors such as UHRF1 are thus barriers between self-replicating cancer cells and the terminal-differentiation fates intended by their transcription factor content.

Apoptosis independent therapies are needed in PDAC. Biomarkers and molecular targets of differentiation-based therapies are identified whose mechanism of action (MOA) suppresses malignant proliferation without engaging cellular apoptosis in either normal or tumor cells, revealing that the most vital methods to antagonize self-replication of multicellular organisms is by differentiation cell cycle exits. Loss of differentiation is crucial for PDAC-genesis, observed by morphological and gene expression changes of pancreas differentiation factors. Differentiation factors that are TSG were epigenetically silenced but not mutated.

Thus, cell differentiation-based therapies for treatment of PDAC are evaluated by effects of identified biomarkers that are transcriptional gene repressors. UHRF1 is a critical repressor protein in PDAC whose epigenetic activity is mediated by its interaction with DNMT1 that drives aberrant DNA methylation and contributes to epigenetic gene silencing of multiple tumor suppressors, including pancreas differentiation factors. Importantly, differentiation factors are epigenetically suppressed, and unlike apoptosis genes are non-mutated and thus available for reactivation.

Cell differentiation is a physiological function that is lost in cancer cells. A pharmacologic method to reactivate suppressed differentiation genes will induce cell differentiation activities upon epigenetic suppression of these factors of differentiation-based cell cycle exits and tissue homeostasis.

Compound DT1, NSC232003, was identified from the NCI developmental therapeutic drug library. DT1 decreased global DNA methylation and activated pancreas differentiation genes, and is a differentiation promoting agent for treating PDAC. DT modulates the epigenetic activity of UHRF1. Cooperation of UHRF1 and DNMT1 produces aberrant epigenetic modifications that deactivate multiple genes, so combined disruption of DNMT1-UHRF1 activity may beneficially modify the aberrant epigenetic changes in PDAC. This approach is innovative from current treatment paradigms (TABLE 1) since the intended MOA is to reactivate non-mutated but epigenetically silenced differentiation genes that are tumor suppressors. Unlike apoptosis treatments (e.g., high dose gemcitabine) that attempt to induce functions of mutated genes (e.g., TP53), epigenetic drugs such as DT1 induce expression of non-mutated factors (e.g., AMY2A/B) that suppress proliferation of cancer cells and perform homeostasis. The unintended cytotoxic effect of most current apoptosis-based therapies is the killing of normal healthy cells. This impairs integrity of the entire organ and the quality of remaining life. Non-apoptosis and cell differentiation-based therapies enhance tissue functions and can be given frequently due to low-dose nature of treatment, thus persistently suppressing tumor volume to increase quality of remaining life.

TABLE 1
Treatment Rational of Current Versus Proposed Therapy

| Current | | Proposed | | |
|---|---|---|---|---|
| Therapy | MOA | Therapy | MOA | |
| (Dose) | (Effect) | (Dose) | (Effect) | Innovation |
| Gemcitabine or | Induce | DT1/DAC | Epigenetic | Low dose |
| Gemcitabine ± | apoptosis | DT1 + | modifications | non-toxic |
| 5-FU/±Cisplatin | (activate | DAC + THU | (activate | activate TSG |
| (high dosage) | TP53) | (low dosage) | silenced TSG) | ↑ homeostasis |

Small molecule nucleoside analogs, such as DT1 and DAC, mimic the natural building blocks of DNA. However, such compounds are prone to metabolic degradation by pathways that regulate nucleoside imbalances, such as pyrimidine metabolizing enzyme cytidine deaminase. Drugs such as DAC have not been applied as solid tissue therapy, perhaps due to fast clearance rate driven by enzymes such as CDA that are highly upregulated in solid tissues. Inhibiting CDA is known to improve DAC efficacy across solid tissue organs, leading to low dose non-cytotoxic therapy. DAC and DT1 are synergistic, with their efficacy substantially improved upon THU treatment using PDAC models (see FIGS. 23, 24, and 26-29).

A therapy combining DT1 and DAC, optionally with THU, will engage epigenetically silenced differentiation genes in PDAC patients. Because KRAS and TP53 alternations yield differentiation-impaired PDAC cells with upregulated UHRF1 activity, many patients could benefit from differentiation-inducing therapy targeting UHRF1 epigenetic activity, including solid tissue malignancies that are recalcitrant to treatment. The apoptosis-independent therapy will provide low dose treatment alternatives with limited cytotoxic effects of therapy, and will enhance tissue homeostasis.

Epigenetic alterations produce gene expression changes that alter the fate of cancer cells. While gene expression is regulated by transcription factors (TF) that are difficult targets for drug development, the epigenetic modifying enzymes such as UHRF1 that mediate the actions of TF are eminently druggable.

In PDAC, the proliferation terminating and pancreas differentiation genes are highly methylated and repressed. One way to repress these genes is by recruiting the repressor enzymes by the transcription factors that regulate their expression. Inhibitors of specific repressor enzymes can restore expression of endogenous proliferation terminating genes, and thus trigger apoptosis-independent cell cycle exit, as clinically validated in liquid tumors, but not in solid tissue malignancies.

The enzymes UHRF1 and DNMT1 are two among many methylation driving enzymes. Upregulation of UHRF1 is a strong predictor of poor survival of PDAC (p<0.0009 n=181 (see FIG. 3). UHRF1 is known to cooperate with DNMT1 to drive DNA methylation, making UHRF1 and DNMT1 molecular targets for epigenetic therapy of PDAC.

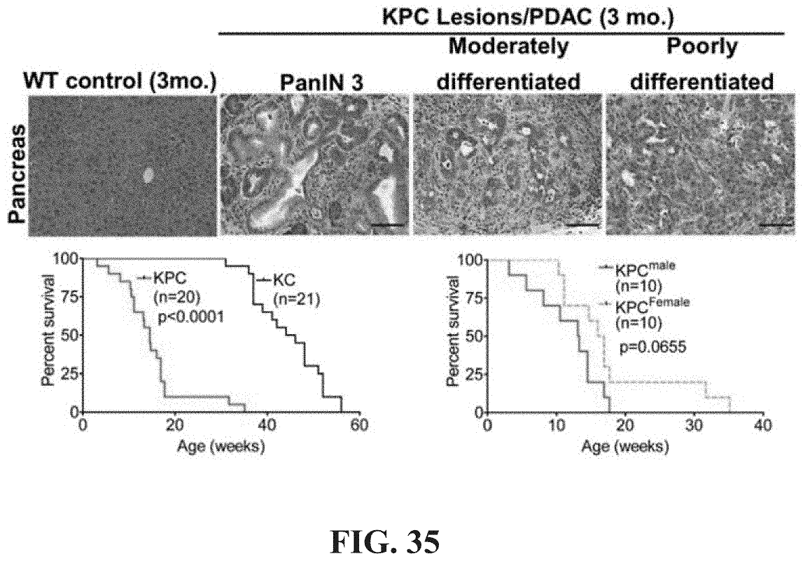

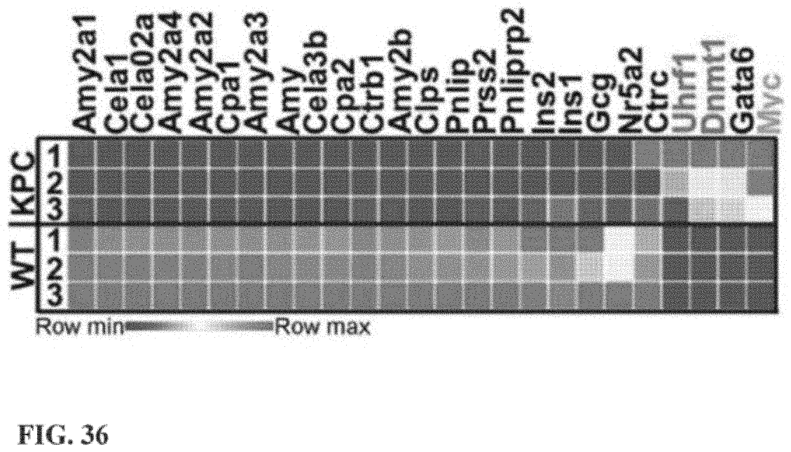

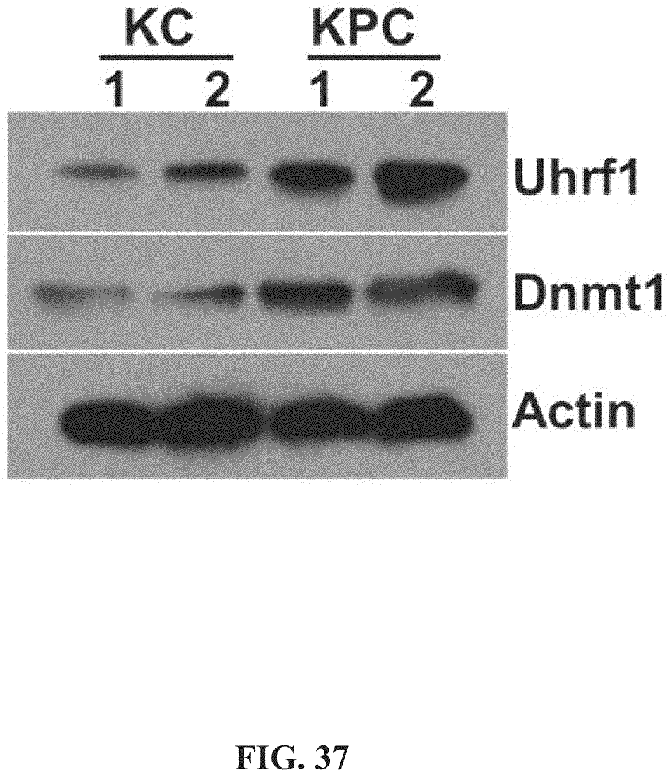

As FIGS. 3 and 6 illustrate, there was high UHRF1 expression in the aggressive PDAC with KRAS and TP53 mutations. As FIGS. 7 and 10 illustrate, pancreas differentiation markers (e.g., NKX6-1, AMY2A/B) were highly methylated and downregulated in high UHRF1-expressing PDAC. Because UHRF1 was highly expressed in KRAS and TP53 mutant cases, a genetically engineered mouse model (GEMM) of PDAC expressing both oncogenic KRAS and mutant TP53 in the pancreas (KPC mice) was used; these mice have accelerated lesion initiation and disease progression with high incidence of metastasis of poorly differentiated PDAC by age 3 months (see FIG. 35). Gene expression RNA sequencing (RNA-Seq) demonstrated that pancreas differentiation genes were downregulated, while Myc mediated proliferation genes were upregulated (FIG. 36). UHRF1 and DNMT1 mRNA and protein were also among upregulated genes (see FIGS. 36 and 37).

There was decreased expression of differentiation factors in human PDAC (n=45) compared to adjacent normal pancreas (n=45) (see FIGS. 11 and 12). Differentiation factors are not mutated in primary PDAC. UHRF1 is known to recruit DNMT1 to enhance maintenance methylation at hemi-methylated DNA, a repressive epigenetic event that silences TSG. The DNA binding SRA domain of UHRF1 recruits DNMT1 to drive DNA methylation. UHRF1 and DNMT1 epigenetically suppress differentiation factors in treatment recalcitrant solid tumors. Thus, anti-corepressor therapy disrupting DNMT1 and UHRF1 can reactivate epigenetically silenced differentiation genes.

Using sh-RNA gene silencing of UHRF1 and DNMT1, there was decreased PDAC cell proliferation, with the strongest suppression seen in sh-UHRF1 transfected cells (see FIGS. 15 and 16). UHRF1 inactivation also decreased global DNA methylation in PDAC cells analyzed by LINE1 global DNA methylation (see FIG. 17).

Small molecule compound NSC232003, DT1, identified from the NCI developmental therapeutics library, decreased DNA methylation and upregulated expression of epigenetically silenced TSG to induce cell morphological changes (increased cytoplasm and decreased nucleus) consistent with epithelial differentiation (see FIGS. 18, 20, and 21). As a nucleoside analog, DT1 efficacy was limited by high CDA expression in the pancreas. CDA was inhibited by THU (1 μM) followed by non-cytotoxic DT1 treatment (0.5 μM). This treatment decreased PDAC proliferation further, increased expression of pancreas differentiation genes, and decreased global DNA methylation (see FIGS. 18, 20, 21, and 26-29). Decitabine (DAC) is a clinically available drug that degrades DNMT1 to decrease DNA methylation. DT1 and DAC efficacy were evaluated in PDAC, and there was potent synergistic effect that decreased PDAC growth and induced differentiation-based morphological changes. Results are shown in FIGS. 20 and 27-29. These synergistic effects were most potent when THU, DT1, and DAC were combined (see FIGS. 27-29).

DT1 may target the SRA domain of UHRF1 to inactivate DNA methylation; optimization and target engagement will identify DT1 molecular targets and establish the source of DT1 therapeutic activity including in vitro and in vivo ADME studies.

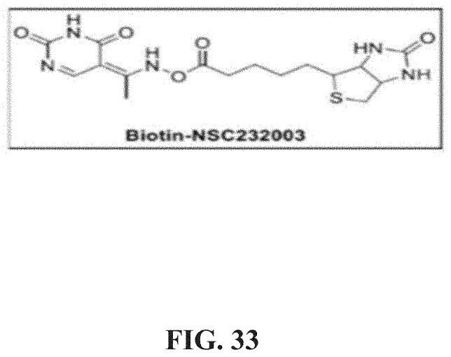

The SRA domain of UHRF1 drives base flipping on unmethylated cytosine, and recruits DNMT1, which drives CpG methylation. DT1 likely interacts with the SRA domain of UHRF1 to modulate its base-flipping dynamics, decreasing DNA methylation. DT1 is conjugated with biotin-linker at the free —OH group of DT1 (shown in FIG. 33) using the PerKit bioconjugation custom service (CellMosaic, www.cellmosaic.com). Biotin-DT1 conjugate is incubated in protein lysates from PDAC cells and covalently crosslinked using biotin streptavadin beads. This interaction will pull down DT1 interacting proteins identified by unbiased liquid chromatography tandem mass spectroscopy (LC-MS/MS) and confirmatory Western blots. Data, searched against a defined database, produce label-free quantitative data (MaxQuant proteomics platform, Indiana University Proteomics Core facility). Experiments are performed in independent biological triplicates.

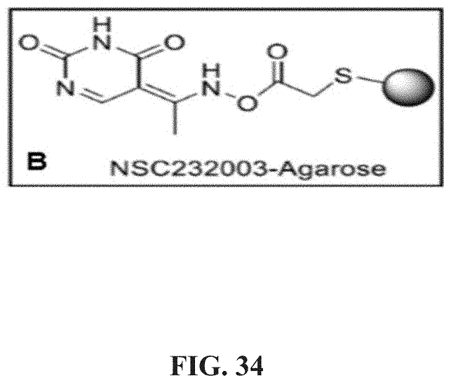

The UHRF1-SRA domain amino acids (435-586) regulating DNA methylation are known; wild-type UHRF1 tagged with tobacco etch virus (TEV) (UHRF1-TEV) were developed with a protease cleavage site. A mutant SRA domain of UHRF1-TEV protein generated by site-directed mutagenesis, and wild-type UHRF1 is incubated with DT1-immobilized to agarose beads (FIG. 34), followed by affinity pull down and TEV cleavage. Bound/unbound DT1-UHRF1 interactions are separated using gel electrophoresis. Specific protein-drug interactions of DT1 are generated in an unbiased approach; identified molecular targets are reevaluated to clarify the MOA and potential SAR properties to improve DT1 efficacy. A potent DT1 molecule will undergo ADME analysis with strong evidence of an established molecular target and MOA.

Specific molecular targets of DT1 and other methyltransferases, such as DNMT1, will be identified based on demonstrated decreases in DNA methylation upon DT1 treatment (FIG. 26). DT1 effects on these enzymes are evaluated by highly sensitive and quantitative commercial assays (Enzo Life Sciences, Product No. ENZ-45016), and/or biochemical assays. If DT1-kinase interactions are the MOA, kinase binding assays will determine DT1 binding affinity to specific kinases using, e.g., a homogeneous binding assay where the displacement of an active site-dependent probe by the drug candidate DT1 is measured by a change in luminescence signal.

DT1 and THU-DT1 are assessed for ADME, in vivo and in vitro, using whole cell pooled human and mouse hepatocyte incubation assays to measure the rate of DT1 disappearance over time. Experiments are performed in triplicate. Pooled hepatocytes are incubated with DT1 (2 μM) independently or with THU (2 μM) followed by metabolic stability assays to estimate intrinsic clearance over time using standard time points of 1-4 hours. DT1 disappearance rate over time is determined, along with the effect of DT-THU incubation.

Mouse in vivo Rapid Assessment of Compound Exposure (RACE) screening determines pharmacokinetic attributes of DT1 in the presence and absence of THU. RACE efficiently estimates pharmacokinetic parameters of exposure of novel chemical probe compounds in mice. Mice in two treatment groups (n=3/group, standard sample size) are intraperitoneally injected with increasing doses of DT1 (0.1-10 mg/kg in group 1, 40 mg/kg in group 2 (selected based on preliminary observations of THU followed by 0.1-10 mg/kg DT1). Plasma is isolated at 2, 6, 15, and 24 hours and analyzed by LC-MS/MS to detect DT1. DT1 clearance rate in group 1 is expected to be high and undetectable especially at low drug concentrations. The clearance rate is improved by CDA inhibition in group 2 using THU, leading to detectable and durable plasma DT1 even at low DT1 concentrations. The lowest detectable and durable concentrations, i.e., detected at all measured time points (e.g., 0.2-5 mg/kg) are used for downstream therapy. Slightly higher but non-cytotoxic concentrations (e.g., 5 mg/kg-30 mg/kg) are used if DT1 is not detected. If DT undergoes too rapid clearance to yield therapeutic efficacy, SAR properties of DT1, or other compounds in the DT1 family, such as NSC81180 and NSC34716, are modified to improve efficacy.

Methods for pharmacologic evaluation of in vivo differentiation induction are developed using in vivo dose-range findings from NSG mice transplanted with ASPC-1 (from female PDAC patient) and MiaPaca-2 (from male PDAC patient) cells. Low-dose (e.g., 0.2 mg/kg) treatments are administered frequently (three times weekly for four weeks). NSG xenografts will be grouped into 5 animals per each treatment group (female animals are transplanted with ASPC-1, and male animals with MiaPaca-2) and treated with low dose and durable DT1 concentrations identified from pharmacodynamics studies (e.g., 0.1-5 mg/kg) in combination with 40 mg/Kg of THU to inhibit CDA. Four therapeutics, DT1, DAC, THU and gemcitabine (standard of care) will likely be used, with at least three repetitions. Animals will be treated on three consecutive days/week for up to 3 weeks by intraperitoneal (IP) injections of low-dose DT1 (concentrations selected after PK studies), 0.2 mg/kg DAC, 40 mg/kg THU, and 60 mg/kg gemcitabine. Tumor growth kinetics are determined by quantifying tumor volumes twice weekly to determine effective in vivo doses that suppress PDAC growth. Low and effective doses are used in pre-clinical evaluation using the KPC model.

Differentiation therapy in a metastatic primary PDAC model using KPC mice are determined by treating KPC male and female animals using low-dose differentiation inducing drugs of DT1 and DAC. DAC is known to induce differentiation cell cycle exits at 0.2 mg/kg when combined with 40 mg/kg THU. DT1 doses are established as previously described. Both male and female KPC mice get tumor lesions starting at 6-8 weeks and 100% of KPC mice have PDAC by 3 months (see FIG. 35). Treatment start at 8 weeks and treatment schedule are adjusted by pilot experiments with pharmacodynamics measurements as previously described. Tumor lesions are measured twice weekly using ultrasound imaging during treatment. Responsive and non-responsive tumors are evaluated before and after relapse. At the end-point (increased mortality of control mice), DNA, RNA and protein are isolated in responder and non-responder animals for down-stream studies using next generation sequencing for epigenetic modifications (changes in DNA methylation), gene expression changes by RNAseq, and protein changes by Western blot in control versus drug treated animals. Pancreas markers such as AMY2AB will be stained by hematoxylin and eosin staining and immunostaining.

Differentiation induction suppresses PDAC growth. Physiologically, proliferating pancreatic precursors stop dividing by undergoing lineage commitment and terminal differentiation. Elevated expression of epigenetically silenced tumor suppressor genes that are differentiation factors of the pancreas (see FIGS. 7 and 10) are anticipated upon DT1 and DAC treatment. Unlike apoptosis therapy that kills normal cells, differentiation therapy will increase expression of pancreas genes in both normal pancreas and PDAC tumor cells. This will enhance tissue function in normal cells (non-cytotoxic), while inactivating tumor growth. Differentiation induction that increases functional pancreas enzymes will be quantified using assays such as amylase assays in-vitro and in-vivo. Failure of PDAC cells to differentiate into pancreatic lineages is also failure to specialize into cells with various pancreatic functions. Differentiation therapy will lead to re-expression of pancreatic enzymes such as amylase and other digestive enzymes, thus enhancing tissue function in healthy cells (see FIG. 18). Global gene expression changes will be generated using the RNA seq studies, and confirmatory studies using gene expression q-PCR will be used.

Xenograft models of PDAC that are highly reproducible and relevant may be used as alternatives to the KPC animal model. Moreover, the KRAS only (KC mice) model produces less aggressive PDAC by 6 months, so 6-month-old animals may be an alternative model.

Should DT1 and DAC be ineffective PDAC therapy, because ATRA and decitabine also induce differentiation in other cancers, the combination including DT1, decitabine, and ATRA will be evaluated using the PDAC models as alternative differentiation therapy, potentially additional compounds that decrease DNA methylation during the screen where DT1 was the most potent. These may be validated, and continued design of drug-screening assays conducted to identify alternative drugs.

All biological experiments are performed in triplicate. The sample size for animal studies is 10 animals per group, adequate power (>0.80) for efficacy determination is expected assuming 0.5 log-fold differences between the compared groups (vehicle and experimental treatment). Sample size calculations are modified to reflect generated data and more accurate estimation of treatment effects and uncertainty. Two independent PDAC xenograft models are used to substantiate translational relevance to human disease. Analysis is performed using PRISM and JMP software. R-programming for modeling and analysis of large datasets such as next gene seq studies is used.

### REFERENCES

Each of the following documents is hereby expressly incorporated by reference herein in its entirety.
1. Rawla et al. Epidemiology of Pancreatic Cancer: Global Trends, Etiology and Risk Factors. World J Oncol 10, 10-27, doi:10.14740/wjon1166 (2019).
2. Von Hoff et al. Increased survival in pancreatic cancer with nab-paclitaxel plus gemcitabine. N Engl J Med 369, 1691-1703, doi:10.1056/NEJMoa1304369 (2013).
3. Rahma et al. Second-line treatment in advanced pancreatic cancer: a comprehensive analysis of published clinical trials. Ann Oncol 24, 1972-1979, doi:10.1093/annonc/mdt166 (2013).
4. Rahib et al. Projecting cancer incidence and deaths to 2030: the unexpected burden of thyroid, liver, and pancreas cancers in the United States. Cancer Res 74, 2913-2921, doi:10.1158/0008-5472.CAN-14-0155 (2014).
5. Ciliberto et al. Role of gemcitabine-based combination therapy in the management of advanced pancreatic cancer: a meta-analysis of randomised trials. Eur J Cancer 49, 593-603, doi:10.1016/j.ejca.2012.08.019 (2013).
6. Sun et al. Does gemcitabine-based combination therapy improve the prognosis of unresectable pancreatic cancer? World J Gastroenterol 18, 4944-4958, doi:10.3748/wjg.v18.i35.4944 (2012).
7. Biankin et al. Pancreatic cancer genomes reveal aberrations in axon guidance pathway genes. Nature 491, 399-405, doi:10.1038/nature11547 (2012).
8. Enane et al. Differentiation therapy and the mechanisms that terminate cancer cell proliferation without harming normal cells. Cell Death Dis 9, 912, doi:10.1038/s41419-018-0919-9 (2018).
9. Velcheti et al. Higher-Level Pathway Objectives of Epigenetic Therapy: A Solution to the p53 Problem in Cancer. Am Soc Clin Oncol Educ Book 37, 812-824, doi:10.14694/EDBK_174175 (2017).
10. Srivastava et al. The Amphimedon queenslandica genome and the evolution of animal complexity. Nature 466, 720-726, doi:10.1038/nature09201 (2010).
11. Domazet-Loso and Tautz. Phylostratigraphic tracking of cancer genes suggests a link to the emergence of multicellularity in metazoa. BMC Biol 8, 66, doi:10.1186/1741-7007-8-66 (2010).
12. Liu et al. UHRF1 targets DNMT1 for DNA methylation through cooperative binding of hemi-methylated DNA and methylated H3K9. Nat Commun 4, 1563, doi:10.1038/ncomms2562 (2013).
13. Guan et al. The epigenetic regulator UHRF1 promotes ubiquitination-mediated degradation of the tumor-suppressor protein promyelocytic leukemia protein. Oncogene 32, 3819-3828, doi:10.1038/onc.2012.406 (2013).
14. Kong et al. Defining UHRF1 Domains that Support Maintenance of Human Colon Cancer DNA Methylation and Oncogenic Properties. Cancer Cell 35, 633-648 e637, doi:10.1016/j.ccell.2019.03.003 (2019).
15. Jansen et al. Deoxyuridine analog nucleotides in deoxycytidine analog treatment: secondary active metabolites? Fundam Clin Pharmacol 25, 172-185, doi:10.1111/j.1472-8206.2010.00823.x (2011).
16. Myrianthopoulos et al. Tandem virtual screening targeting the SRA domain of UHRF1 identifies a novel chemical tool modulating DNA methylation. Eur J Med Chem 114, 390-396, doi:10.1016/j.ejmech.2016.02.043 (2016).
17. Hingorani et al. Trp53R172H and KRASG12D cooperate to promote chromosomal instability and widely metastatic pancreatic ductal adenocarcinoma in mice. Cancer Cell 7, 469-483, doi:10.1016/j.ccr.2005.04.023 (2005).
18. Mueller et al. Evolutionary routes and KRAS dosage define pancreatic cancer phenotypes. Nature, doi:10.1038/nature25459 (2018).
19. Saunthararajah et al. Evaluation of noncytotoxic DNMT1-depleting therapy in patients with myelodysplastic syndromes. J Clin Invest 125, 1043-1055, doi:10.1172/JCI78789 (2015).
20. Enane. Hepatocyte differentiation and hepatocellular carcinoma: Rationale for p53 independent therapy PhD thesis, Case Western Reserve University, (2017).
21. Enane et al. GATA4 loss of function in liver cancer impedes precursor to hepatocyte transition. The Journal of Clinical Investigation 127, doi:10.1172/JCI93488 (2017).
22. Gu et al. Leukemogenic nucleophosmin mutation disrupts the transcription factor hub regulating granulo-monocytic fates. The Journal of Clinical Investigation, doi:10.1172/JC197117 (2018).
23. Morton et al. Mutant p53 drives metastasis and overcomes growth arrest/senescence in pancreatic cancer. Proc Natl Acad Sci USA 107, 246-251, doi:10.1073/pnas.0908428107 (2010).
24. Ng et al. p53 independent epigenetic-differentiation treatment in xenotransplant models of acute myeloid leukemia. Leukemia 25, 1739-1750, doi:10.1038/leu.2011.159 (2011).
25. Negrotto et al. Noncytotoxic differentiation treatment of renal cell cancer. Cancer Res 71, 1431-1441, doi:10.1158/0008-5472.CAN-10-2422 (2011).
26. Lo-Coco et al. Retinoic acid and arsenic trioxide for acute promyelocytic leukemia. N Engl J Med 369, 111-121, doi:10.1056/NEJMoa1300874 (2013).
27. Hu et al. RUNX1 regulates corepressor interactions of PU.1. Blood 117, 6498-6508, doi:10.1182/blood-2010-10-312512 (2011).
28. Gu et al. Runx regulation of Pu.1 corepressor/coactivator exchange identifies specific molecular targets for leukemia differentiation therapy. J Biol Chem 289, 14881-14895, doi:10.1074/jbc.M114.562447 (2014).
29. Vazquez et al. The genetics of the p53 pathway, apoptosis and cancer therapy. Nat Rev Drug Discov 7, 979-987, doi:10.1038/nrd2656 (2008).
30. Lavelle et al. Effects of tetrahydrouridine on pharmacokinetics and pharmacodynamics of oral decitabine. Blood 119, 1240-1247, doi:10.1182/blood-2011-08-371690 (2012).
31. Kilin et al. Dynamics of Methylated Cytosine Flipping by UHRF1. J Am Chem Soc 139, 2520-2528, doi:10.1021/jacs.7b00154 (2017).
32. Li et al. Structural and mechanistic insights into UHRF1-mediated DNMT1 activation in the maintenance DNA methylation. Nucleic Acids Res 46, 3218-3231, doi:10.1093/nar/gky104 (2018).
33. Weisenberger et al. Analysis of repetitive element DNA methylation by MethyLight. Nucleic Acids Res 33, 6823-6836, doi:10.1093/nar/gki987 (2005).
34. McAnally et al. In Vivo Rapid Assessment of Compound Exposure (RACE) for Profiling the Pharmacokinetics of Novel Chemical Probes. Curr Protoc Chem Biol 4, 299-309, doi:10.1002/9780470559277.ch120072 (2012).
35. Hidalgo et al. Patient-derived xenograft models: an emerging platform for translational cancer research. Cancer Discov 4, 998-1013, doi:10.1158/2159-8290.CD-14-0001 (2014).

What is claimed is:

1. A method of treating pancreatic cancer in a patient in need thereof, the method comprising administering to the patient an effective amount of a pharmaceutical composition comprising NSC232003 (DT1), NSC127716 (DAC), and one or more pharmaceutically acceptable carriers, excipients, or diluents.

2. The method of claim 1, further comprising administering NSC112907 (THU) to the patient.

3. The method of claim 1, wherein the route of administration is parenteral.

4. The method of claim 1, wherein the patient is a human.

5. The method of claim 1, further comprising administering to the patient at least one additional active ingredient.

6. The method of claim 1, wherein the amount of DT1 administered to the patient is from about 0.1 to about 10 mg of DT1 per kg of patient body weight.

7. The method of claim 6, wherein the amount of DAC administered to the patient is from about 0.1 to about 1 mg of DAC per kg of patient body weight.

8. The method of claim 2, wherein the amount of THU administered to the patient is from about 10 to about 40 mg of THU per kg of patient body weight.

9. The method of claim 1, wherein the patient is administered from about 0.1 to about 10 mg of DT1 per kg of patient body weight and from about 0.1 to about 1 mg of DAC per kg of patient body weight.

10. The method of claim 2, wherein the patient is administered from about 0.1 to about 10 mg of DT1 per kg of patient body weight, from about 0.1 to about 1 mg of DAC per kg of patient body weight, and from about 10 to about 40 mg of THU per kg of patient body weight.

11. The method of claim 1, wherein the pancreatic cancer is pancreatic ductal cell adenocarcinoma (PDAC).

12. A method of treating pancreatic cancer in a patient in need thereof, the method comprising administering to the patient an effective amount of an inhibitor of UHRF1 and an inhibitor of DNMT1.

13. The method of claim 12, further comprising administering an inhibitor of cytidine deaminase (CDA).

14. The method of claim 12, wherein the pancreatic cancer is pancreatic ductal cell adenocarcinoma (PDAC).

15. A method of treating pancreatic cancer in a patient in need thereof, the method comprising administering to the patient an effective amount of a pharmaceutical composition comprising NSC127716 (DAC), NSC122758 (ATRA), and one or more pharmaceutically acceptable carriers, excipients, or diluents.

16. The method of claim 15, wherein the route of administration is parenteral.

17. The method of claim 15, wherein the patient is a human.

18. The method of claim 15, wherein the amount of ATRA administered to the patient is from about 1 to about 100 mg of ATRA per kg of patient body weight.

19. The method of claim 18, wherein the amount of DAC administered to the patient is from about 0.1 to about 1 mg of DAC per kg of patient body weight.

20. The method of claim 15, wherein the pancreatic cancer is pancreatic ductal cell adenocarcinoma (PDAC).
